(19) United States
(12) Patent Application Publication
(10) Pub. No.: US 2018/0078579 A1
(43) Pub. Date: Mar. 22, 2018

(54) MICRONUTRIENT FORMULATIONS FOR ENVIRONMENTAL EXPOSURE APPLICATIONS

(71) Applicant: Gerald M. Haase, Denver, CO (US)

(72) Inventor: Gerald M. Haase, Denver, CO (US)

(21) Appl. No.: 15/603,197

(22) Filed: Oct. 1, 2017

Related U.S. Application Data
(63) Continuation-in-part of application No. 12/284,841, filed on Sep. 25, 2008, now Pat. No. 9,655,966.

(51) Int. Cl. A61K 33/30; A61K 31/07; A61K 31/375; A61K 31/355; A61K 31/197; A61K 31/385; A61K 33/04; A61K 31/015; A61K 31/593; A61K 31/51; A61K 31/525; A61K 31/455; A61K 31/675; A61K 31/519; A61K 31/714; A61K 31/4188; A61K 33/06; A61K 33/24; A61K 31/205; A61K 31/122

(52) U.S. Cl. CPC A61K 33/30; A61K 31/122; A61K 31/375; A61K 31/355; A61K 31/197; A61K 31/385; A61K 33/04; A61K 31/015; A61K 31/593; A61K 31/51; A61K 31/525; A61K 31/455; A61K 31/675; A61K 31/519; A61K 31/714; A61K 31/4188; A61K 33/06; A61K 33/24; A61K 31/205; A61K 31/07

(57) ABSTRACT

A radioactive protection micronutrient formulation system is provided and the system comprises: a formulation consisting essentially of antioxidants, the antioxidants are selected from the group consisting essentially of vitamin C, vitamin E, N-acetyl cysteine, natural mixed carotenoids, and alpha-lipoic acid, vitamin A (palmitate), vitamin D-3 (cholecalciferol), thiamine mononitrate, riboflavin, niacinamide ascorbate, d-calcium pantothenate, pyridoxine hydrochloride, cyanocobalamin, folic acid, D-Biotin, selenium (1-seleno-methionine), chromium picolinate, zinc glycinate, calcium citrate and magnesium citrate and mixtures thereof; and plus a booster formulation selected from a group consisting essentially of vitamin C, d-alpha tocopheryl acid succinate, alpha tocopherol, N-acetyl cysteine, natural mixed carotenoids and alpha lipoic acid, the formulation is designed to reduce the risk in humans exposed to doses of ionizing radiation of becoming subjected to at least one condition selected from the group consisting essentially of radiation-induced acute leukemia, breast cancer, thyroid cancer and other somatic and heritable mutations.

### RELATED APPLICATION

This application is a continuation-in part application of U.S. application Ser. No. 12/284,841, entitled “MICRONUTRIENT FORMULATIONS FOR RADIATION EXPOSURE” which was filed on Sep. 25, 2008 and has now has issued has U.S. Pat. No. 9,655,966”.

### BACKGROUND OF THE INVENTION

### Field of the Invention

This invention relates to a use of micronutrient formulations to reduce the effects of environmental exposures on humans. Hazardous environmental exposures are implicated in many adverse health conditions worldwide and antioxidants are protective agents involving many cellular processes that must be considered as targets for therapeutic intervention. The present invention relates to the use of multiple micronutrients including dietary and endogenous antioxidants with glutathione-elevating agents to reduce the adverse health effects on humans during episodic, occupational or accidental exposures to hazardous environmental factors or potentially toxic agents. In addition to an oral mode of consumption, the BioShield formulations may be provided in several alternative platforms including, but not limited to liquids, aerosols, dissolvable disks, injectable forms, those absorbed through the skin, lipid-encased and droplet forms and delayed absorption technologies.

### Description of the Related Art

It is recognized that use of tobacco products contributes to human illness. Recent studies estimate that 14 million major medical conditions among U.S. adults ages 35 and older are attributable to use of tobacco products (2). At present, it is estimated that there are more than 50 million smokers in the United States and public health efforts are continuing to prevent and decrease smoking and chewing of tobacco-related products. Efforts to convert users to Electronic cigarettes (E-cigs) are also underway. However, tobacco products contain tar and nicotine and E-cig vapor contains toxins, such as propylene oxide, a potential carcinogen, acrolein, a pro-inflammatory irritant, nitrosamines, formaldehyde, acetaldehyde, isoprene, acetic acid, 2-butanodione, acetone, propanol, propylene glycol, diacetin, and ultrathin particles of heavy metals. These potentially harmful constituents not only accumulate in the lung, but also in the blood stream and are deposited in other organs including the brain. Antioxidant micronutrient supplementation can target the organ systems of the entire body, and, therefore, this invention offers a rational strategy to potentially reduce the risks associated with smoking products.

There are a number of mechanisms by which tobacco products may cause increased oxidative stress in humans. The following parameters have been measured in smokers and found to be sensitive indicators of injury: (a) increased membrane peroxidation, (b) reduced plasma uric acid levels, (c) increased vascular wall leukocyte adhesion and intravascular leukocyte platelet aggregation, (d) increased monocyte adhesion to endothelial lining, (e) endothelial cell dysfunction, (f) plasma lipoprotein oxidation and (g) increased formation of antibodies to oxidized low density lipoproteins.

There are also important mediators of chronic inflammatory responses which can measure the effects of smoking-related oxidative stress: (a) increased levels of interleukin-6 and tumor necrosis factor, (b) increased plasma levels of oxidized forms of vitamin C such as dehydroxyascorbate, (c) oxidative DNA damage as detected by increased urinary levels of 8-hydroxydeoxyguanosine and (d) increased levels of 8-epi-prostaglandin (PGF-2), thiocyanate and exhaled ethane, all as stable measures of in-vivo lipid peroxidation. The antioxidant micronutrients in this invention have been shown to normalize many parameters of stress.

Because tobacco cigarette smoking causes adverse health effects and cessation is difficult due to nicotine addiction, efforts were made to develop a non-tobacco cigarette. E-cigs contain a rechargeable lithium battery powered device that produced an aerosol vapor resembling tobacco cigarette smoke. The vapor is usually formed by heating a solution of propylene glycol and glycerin with or without nicotine at high temperatures (40-65° C.). Comparative analyses of E-cig vapor and tobacco cigarette smoke show that many common toxic chemical levels are lower or not present in E-cigs. However, some additional toxic chemicals are found in the vapor of E-cigs that are not present in tobacco cigarette smoke. In relation to micronutrients, smoking decreases the plasma levels of vitamin C, beta-carotene, cyanocobalamin, vitamin E and folic acid.

Increased oxidative stress and chronic inflammation produced by the constituents of tobacco products and E-cigs are likely pathways that may contribute to adverse health effects. Therefore, this invention describes the scientific rationale and evidence to suggest that a combination of multiple micronutrients may protect against injurious effects of toxic chemicals in the vapor. Specifically, these oral supplements may provide overall protection against oxidative damage and chronic inflammation produced by tobacco products as well as the oxidizing agents, carcinogens and ultrathin particles of heavy metals present in E-cig vapor.

Multiple antioxidants are important for health maintenance in that they stimulate the immune system, reduce the formation of toxic chemicals in the intestinal tract and reduce the diffusion of cancer forming substances in the liver. They also protect cells from the injurious effects of free oxygen and nitrogen radicals produced in the body through normal biochemical processes (oxidative damage) as well as from acute and chronic inflammation.

Multiple antioxidant micronutrients rather than any single supplement are necessary for an optimal effect. Pharmacokinetic research has shown that less than ten percent of oral micronutrient intake is absorbed and the half-life of water soluble constituents can be as little as six hours while that of lipid soluble micronutrients is about eight hours. Therefore, in order to maintain consistently high serum levels of these substances, it is essential to modify the usual dose schedule as outlined in this invention to enhance the biological effects.

It is established that many environmental, occupational and hazardous exposures are potential mutagens and carcinogens that can induce somatic and heritable mutations, and neoplastic and certain non-neoplastic diseases. Therefore, the strategy of the current invention is to leverage the demonstrated effects of unique micronutrient combinations on oxidative stress, immune function and neuroprotection to provide a benefit against the targeted adverse exposure risks.

Human Studies: Oxidative Damage and Immune Function

This study demonstrated reduction of oxidative damage using malondialdehyde (MDA) concentration in the blood and urine as an index of oxidative stress, and enhancement of immune function, using the test of lymphocyte transformation (TLT) in human volunteer subjects (3).

Material and methods: Two groups of healthy volunteers, Group 1 (20-30 years) and Group 2 (65-74 years) participated. They consumed the micronutrients orally twice a day (morning and evening) before meals for a period of six months. Blood and urine samples were collected every two weeks for a period of two months before supplementation and for a period of six months after supplementation. Plasma and urine were frozen until analysis. MDA levels were determined in plasma and urine samples, whereas TLT values were determined using isolated peripheral lymphocytes. The average value of MDA or TLT before supplementation was used as a control for the subsequent values after supplementation.

Test of lymphoblastic transformation (TLT): The TLT, or lymphocyte transformation assay, is based on the capacity of T-lymphocytes to transform into blast cells when grown in the presence of the antigen phytohemagglutinine (PHA). Microscopic observation allows measurement of the level of blastic transformation. The normal baseline values of blastic transformation ranged from 60-65% of the untransformed lymphocytes.

Effect on immune function: The TLT value was used as a measure of overall immune function. The results showed that the average TLT value increased in Group 1(younger subjects) as well as in Group 2 (older subjects) receiving the supplement in comparison to that observed before taking the supplement (FIG. 1). This increase was slightly more pronounced in Group 1 (FIG. 1A) than in Group 2 (FIG. 1B). A summary of the average value of MDA levels in the plasma and urine, and TLT is presented in FIG. 2.

Results: The results showed that daily oral supplementation gradually decreased MDA concentrations in the blood and urine, whereas it gradually increased the value of TLT over a six month period. The response of Group 1 (young subjects) was slightly greater than that of Group 2 (older subjects). This decrease in the oxidative damage parameter and a increase in the immune function parameter occurred in all subjects and was sustained and still improving throughout the entire study period.

These results are the first demonstration to show that oral supplementation with a proprietary multiple micronutrient preparation gradually reduces oxidative damage and improves immune function in human subjects. This study further shows that the effects of the antioxidant supplement on oxidative damage and immune function are slightly more pronounced in younger populations.

Oxidative damage impact: Prospective, randomized, double-blind, placebo-controlled trial in U.S. Marine Corps volunteers subjected to cold, high altitude and exertion stress at the Mountain Warfare Training Center, 29 Palms, Calif. (4).

The study group consumed an active formulation and the control group received a placebo supplement for duration of the 12-week training course. Serial urine samples were taken from all participants and analyzed before and after supplementation for sensitive markers of oxidative damage (8-hydroxyguanosine). Safety was assessed by field reports of adverse reactions.

In the placebo group, 42% of subjects had low levels of the biological marker before supplementation, and the remaining 58% exhibited high levels of oxidative damage reflecting the extreme conditions. After consuming the placebo, only 25% of the subjects still had low levels of oxidative damage and 75% had high levels, demonstrating 17% deterioration in oxidative status.

Conversely, in the antioxidant treated group, 30% of the subjects had low levels of 8-hydroxyguanosine before supplementation and 70% exhibited high levels of the marker. After receiving the active formulation, 71% of the subjects showed low oxidative damage levels and only the remaining 29% had high levels, demonstrating 41% improvement in oxidative status (table). This documented that the formulation not only prevented more oxidative stress from occurring during extreme environments but, in fact, reduced the oxidative damage that was already present.

In addition, the changes in plasma levels of antioxidant micronutrients were also documented. The placebo group showed no difference in the levels of alpha-tocopherol (vitamin E) in the blood, before (4.8 ug.ml) or after (4.6 ug/ml) supplementation. However, the average value almost doubled (4.1 ug/ml to 7.6 ug/ml) in the antioxidant treated group, documenting that the formulation is absorbed well even in the face of extreme conditions and intense activity. There were no reported or observed adverse effects from consuming the formulation.

TABLE

| | | | Military Antioxidant | |
|---|---|---|---|---|
| | Placebo | | Formulation | |
| | Pre-treatment | Post- treatment | Pre-treatment | Post-treatment |
| | N = 12 | N = 12 | N = 17 | N = 14 |
| | (% of total subjects) | | (% of total subjects) | |
| Low value | 42 | 25 | 30 | 71 |
| High value | 58 | 75 | 70 | 29 |

The 8-hydroxuguanosine level (μg/mg creatine) up to 2.0 was considered low value (low oxidative damage) and 2.1 to 3.0 as high value (high oxidative damage).). The number of subjects with high oxidative damage increased after placebo consumption. However, the number of subjects with high oxidative damage decreased after antioxidant treatment. N refers to the number of subjects in each group.

Neuroprotection: Prospective, randomized, double-blind clinical trial in U.S. Marine Corps personnel suffering mild to moderate concussive brain injury (blasts) after returning from combat in Iraq (5). nAll patients had received their injury 3-20 weeks prior to study entry.

The control group received standard rehabilitation (steroids, physical therapy, vestibular rehabilitation and supportive care) for 12 weeks.

The study group received standard care plus the formulation (two capsules by mouth twice per day) for the same time period.

All patients were evaluated by the same outcome measures that included the Sensory Organization Test (SOT) by Computerized Dynamic Posturography (CDP), Dynamic Gait Index (DGI), the Activities Balance Confidence (ABC) scale, Dizziness Handicap Index (DHI), Vestibular Disorders Activities of Daily Living (VADL) score, and the Balance Scoring System (BESS) test. The therapist who performed and graded the testing was blinded as to whether or not the patient was in the control group or receiving antioxidants. The pre-trial test scores did not differ significantly between the two groups on any of the tests.

The study group receiving the micronutrient formulation demonstrated more rapid and complete recovery than did the control group even though the formulation was not consumed until well after the concussions were suffered. Postural stability, dynamic gait index, and dizziness handicap scores were already significantly different after only 4 weeks, and this improvement grew in significance by the end of 12 weeks. The Sensory Organization Test score by computerized dynamic posturography was 78 for the antioxidant group as compared to 63 for the control group (P<0.05).

The improvement noted in the micronutrient group on the other tests also trended to a greater degree than that of the control group. Questionnaires were also administered regarding energy levels, exercise, and overall cognitive issues by an investigator who did not know to which group the patient belonged. The study group demonstrated a significant increase in energy level, exercise tolerance and cognitive ability at every weekly time point. There were no adverse effects from the formulation.

It is well established that exposure to radiation is a potent mutagen and carcinogen that can induce somatic and heritable mutations, and neoplastic and certain non-neoplastic diseases. However, this modality is also used as a medical device in the diagnosis and treatment of human diseases. Children are more sensitive than adults to ionizing radiation with respect to all criteria of radiation damage, including cancer. Also, the time interval between radiation exposure and death in children is longer than in adults, which would increase the risk of expression of deleterious effects in the young (6, 7). No doses of ionizing radiation are considered totally safe.

The growing use of these X-ray-based devices has raised concerns about potential hazards of such procedures in increasing the risk of cancer and somatic and heritable mutations in individuals receiving diagnostic doses of radiation. These risks also exist in radiation workers in health care and those in government (nuclear) and military occupations, or those residing near such facilities and who are exposed to higher doses of ionizing radiation per year than non-radiation workers. The number of radiation workers has increased proportionally with increased diagnostic radiation procedures and use of nuclear energy. In 2008, it was estimated that over 60 million CT scans were performed in the U.S. (8). This estimate did not include other diagnostic procedures such as chest x-rays, dental x-rays, fluoroscopic imaging, positron emission tomography and other nuclear medicine scans. Because of the potential health hazards, developing an effective strategy that involves both physical and biological protection methods against potential damage from radiation is relevant and constitutes an important target for this invention.

Efforts to develop protection against radiation damage began soon after the discovery of X-rays in 1895 by Dr. Roentgen, a German scientist. However, the observation by Dr. Muller of Columbia University, in 1927, that x-ray causes gene mutations in Drosophila melanogaster (common fruit fly) provided new impetus to develop an effective physical and biological protection against radiation damage. The initial physical concept of radiation protection involved three principles that can reduce dose levels: (a) lead-shielding of unexposed areas, especially radiosensitive organs such as bone marrow, intestine, gonads and thyroid; (b) increased distance between the radiation source and radiation workers or patients; and (c) reduction of radiation exposure time. These principles are useful but have limitations. For example, during fluoroscopy, it may not be possible to protect the gastrointestinal tract (one of the most radiosensitive organs) against radiation damage by lead shielding alone. Increasing the distance between the radiation source and individuals to be exposed may not be practical for many radiation workers, patients, civilian or military personnel. Reducing radiation exposure time is mostly pertinent only for those that care for patients who have received radioisotopes for medical purposes or those who are responsible for radioactive decontamination as a result of nuclear accidents or attack. To address the growing concerns of radiation-induced damage, the concept of ALARA (as low as reasonably achievable) with respect to dose was recommended by national and international radiation protection agencies for radiation workers and individuals receiving diagnostic doses of radiation (9). Additional recommendations are being made to reduce the number of diagnostic medical procedures whenever possible in order to reduce the total dose exposures (10-12). These recommendations and physical principles do not provide a biological protection strategy for those who justifiably receive low-dose radiation during diagnostic procedures, for radiation workers in many occupations who are exposed daily as well as for the general public who may be episodically exposed. Increasing awareness about the higher risk of cancer among these individuals is of concern to health care experts (13, 14).

Therefore, the issue of this potential biological protection has drawn attention from radiobiologists and clinicians. In addition, the threat of terrorism, nuclear power plant accidents, weather catastrophes and other environmental causes, the prevention and mitigation before and after radiation exposure is of significant interest to public policy makers.

The BioShield formulations are based on attenuation of biochemical factors that initiate (free radicals) and promote (free radicals and pro-inflammatory cytokines) radiation damage in humans. The individual and combined constituents have been shown to be of radioprotective value in laboratory experiments (tissue culture and animal models) and human studies. The doses of antioxidants employed in these formulations have been used by the public for decades without any reported toxicity, and may be generally regarded as safe for human consumption. They are considered food supplements and, therefore, require no FDA approval or medical prescription for human usage. Published data show that these formulations are effective against episodic low doses of radiation, such as for individuals listed in Section 1.1 (e.g. those receiving medical imaging and frequent flyers; BioShield R1), and those listed in Section 2.1 (e.g. radiation workers and constant cellular phone users; BioShield R2). In addition, a specific formulation, BioShield R3, in combination with standard therapy, may be useful in decreasing adverse effects in individuals with catastrophic exposures (Section 3.1).

Dose estimates: The typical radiation doses for an adult from a chest CT scan can range between 6-10 milliSieverts (mSv) (15). Several radiation dose estimates for imaging studies in adults and children have been published (15, 16). The effective radiation dose estimates from average diagnostic procedures are presented in Table 1, and other dose estimates from specific diagnostic procedures are described in Tables 2 and 3. The average annual dose from background radiation in the U.S. is approximately 3 mSv.

High-dose effects: Radiation doses below 2 Sieverts (Sv) are not likely to produce any mortality, but the survivors will have increased risk of neoplastic and non-neoplastic diseases. Doses between 2.7 and 5.0 Sv produce 50-100% mortality within 60 days from bone marrow syndrome in humans. Doses between 6.0 and 40 Sv cause 100% mortality within 14 days from gastrointestinal (GI) syndrome. Doses 50 Sv and above produce 100% mortality within 24 hours from central nervous system (CNS) syndrome.

Extensive scientific reviews have been published in support of the linear no-threshold model for radiation-induced cancer (13, 14). A dose of from a CT scan may increase the risk of cancer in children by 1 in 1000 exposed individuals (10, 17, 18). A study has also reported that the lifetime cancer mortality risks for a one-year old child exposed to the radiation dose from a CT scan are 0.18% (abdominal scan) and 0.07% (head scan). This risk estimate is an order of magnitude higher than for adults. It was further estimated that in 2001, approximately 600,000 abdominal and head CT scans were performed in children under the age of 15 years, and that 500 of the exposed children might die from cancer attributed to the scan (19). A Canadian study reported that an abdominal CT study in a 5-year old child, may increase lifetime risk of radiation-induced cancer by approximately 26.1 per 100,000 in female and 20.4 per 100,000 in male patients (20). A study performed in Israel estimated an increase of about 0.29 percent over the total number of patients who are eventually are expected to die from cancer (12). Since children and adults receiving diagnostic doses of radiation and radiation workers may also be exposed to environmental, chemical and biological carcinogens, as well as common tumor promoters (e.g. caffeine, viruses, ozone) that enhance radiation-induced cancer risk during their lifetime, the actual overall cancer mortality risk is likely higher than estimated. The cancer risks after low doses of radiation are presented in Table 4.

The dose-estimates and cancer risk from cardiovascular imaging have been published (21). It has been estimated that about five billion imaging examinations are performed worldwide each year, and two-thirds involve ionizing radiation (22). In 2006, the estimated medical radiation exposure dose in the U.S. had reached 3.2 mSv (21) which is more than six-fold higher than that estimated in 2004 (23). The analysis of the risk of cancer on the basis of the annual number of diagnostic x-rays taken in UK and 14 other developing countries revealed the cumulative risk varied from 0.6 to 1.8%, whereas in Japan, which used the highest number of annual diagnostic x-rays, it was more than 3% (23). Cardiologists prescribe and/or directly perform greater than 50% of radiation imaging examinations that contribute to about two-thirds of the total effective dose to patients (24). It has been estimated that about 20 million nuclear medicine examinations were performed in 2006, and cardiac examinations accounted for about 57% of these procedures and 85% of the radiation dose (25). Laboratory studies also support the risks from this significant use of imaging technology since radiation exposure during interventional cardiovascular procedures induces damage to DNA and causes chromosomal aberrations (26). The U.S. government's Biological Effects of Ionizing Radiation Committee VII report estimated that a dose of 15 mSv may increase cancer risk by 1 in 750 cases. Others have estimated that coronary multi-slice CT that delivers about 20 mSv may increase the risk of cancer by 1 in 500 patients, and coronary stenting that delivers 25 mSv by 1 in 400 (27, 28).

Dental x-rays are essential for evaluating dental problems. However, studies have shown that dental x-rays induce formation of micronuclei (breaking of the cell nucleus into small fragments) in the oral cavity in both adults and in children, when these cells are examined under the microscope soon after x-ray imaging (29, 30). This micronucleus assay in exfoliated buccal cells is considered a useful and minimally invasive assay method for monitoring genetic damage in humans (31). While the long-term significance of this observation is unknown, even very small radiation doses can cause measurable damage in cells soon after irradiation. A 2010 epidemiologic study was published in the journal Acta Oncologica by Dr. Anjum Memon of Brighton and Sussex Medical School in England and his collaborators at the University of Cambridge and Kuwait University. This revealed that the risk of thyroid cancer increased by about two-fold in men and women who received four dental x-rays, by about four-fold in those who received 5-9 dental x-rays, and 5.4-fold in those who received 10 or more dental x-rays.

Mobile cellular and smart phone technology and its use have exploded during last decade throughout the world. It is estimated that about 4-5 billion people currently use mobile phones. The fact that radiofrequency electromagnetic fields (RF-EMF) from the cellular phones can be absorbed into the brain has prompted concerns that constant use of these phones for a long period of time may increase the risk of acoustic neuroma and other brain tumors. RF-EMF exposure (at common frequency ranges of 30 kHz to 300 GHz) is ubiquitous since sources can be from personal devices, occupational (e.g. heaters, radar) and environmental (e.g. broadcast antennas, medical devices) sources. In addition, heavy mobile phone users show higher levels of oxidative damage from the production of toxic peroxide and free radicals (32). The DNA damage associated with oxidative stress is also thought to adversely affect male reproduction and increase the risk of brain and testicular cancer (33).

The effects of cellular phone use on cancer risk have been investigated by epidemiologic methods and concerns have been raised (34, 35). It appears that the regular heavy use of mobile phones for a period of 10 years or more was associated with increased risk of benign acoustic neuroma as well as malignant glioma (36, 37). There are contrary reports but they generally include assessments after shorter exposure times or propose that the data showing increased risk was confounded by recall bias (38, 39). Another study demonstrated that heavy cellular phone use was associated with an increased risk of parotid gland tumors (40). An epidemiologic study of an Egyptian population living nearby cellular phone base stations showed an increased risk of developing neuropsychiatric problems such as headache, memory changes, dizziness, tremors, depressive symptoms, and sleep disturbance compared to a control population (41).

Conclusions from laboratory studies of cellular technology in animal and cell culture models are limited in assessing human disease. Nevertheless, radiofrequency radiation emitted from a cellular phone produced no effect on cancer incidence in mice while exposure of mammalian cells in culture to 835-MHz radiofrequency radiation electromagnetic field enhanced the levels of chromosomal aberrations induced by a chemical (ethylmethanesulfonate) (42, 43).

Currently, the European and U.S. Federal Aviation Administration as well as the individual airlines consider all flight personnel (pilots, flight crew and flight attendants) to be radiation workers. They are exposed to cosmic ionizing radiation, potential chemical carcinogens (fuel and jet engine exhaust), electromagnetic fields from cockpit instruments, and occupational hazards such as disrupted sleep patterns. Several epidemiologic studies have evaluated the risk of cancer in these populations. Most studies suggest that there is a clinically measurable increased risk of prostate, brain, melanoma and other skin cancers, as well as acute myeloid leukemia in male pilots (44-47), and breast cancer, melanoma (47-50) and bone cancer (48) in female flight attendants. In addition, military pilots had a higher incidence of bladder and testicular cancer than non-flyer military personnel (51). Finally, laboratory analyses from commercial flight personnel showed increased chromosomal abnormalities in flight crews compared to non-flying “control” individuals (52, 53). With such strong clinical evidence and correlative laboratory findings, it seems reasonable to be concerned potential risks in the civilian frequent flyer population who exceed 100,000 miles in high elevation commercial flight annually.

The incidence of non-neoplastic diseases and intermediate health risks measured by specific biochemical markers were studied in children living in radiation-contaminated areas near the 1986 Chernobyl nuclear accident site. The incidence of thyroid gland enlargement and vision disorders, mostly dry eye syndrome, was closely related to the levels of contamination (54). Increased levels of oxidized conjugated dienes, products of lipid peroxidation, were found among these children. Increased levels of spontaneous chemiluminescence, an indicator of enhanced oxygen radical activity, in leukocytes of children living in contaminated areas were also observed (55). Radioactive fallout from Cold War nuclear weapons testing has been reported to be responsible for thousands of cancer deaths in the U.S. and Russia in the past 50 years (56, 57). Adverse effects are also being noted from the 2011 Fukushima Daiichi nuclear power plant meltdown after earthquake/tsunami damage. Elevated levels of airborne beta nuclear radiation were detected in the U.S., especially on the West Coast, and increased cases of congenital hypothyroidism occurred in children born in California after the disaster (58). Similar to risks created by nuclear disasters, the natural occurrence of substances such as radon is also related to serious health effects such as lung cancer (59). Radon has been considered more lethal than carbon monoxide and may cause an increase in skin cancer rates (60, 61).

A clear association between air pollution and multiple health issues related to oxidative stress has been reported (62). The adverse health effects include deaths from heart disease, decreased lung function, increased hospital admission and overall increased mortality. A recent estimate from the World Health Organization attributes about seven million deaths annually to indoor and outdoor air contamination (63). Chronic exposures may lead to stroke, heart disease, obstructive lung disease, respiratory infections and lung cancer. Environmental climate change related to additional heat stress also was associated with asthma, infectious diseases, intestinal conditions and mental stress disorders (64). It has also been demonstrated that pregnant women exposed to significant air pollution are five times more likely to produce children who suffer from behavioral problems such as attention deficit hyperactivity disorder (65). Finally, recent reports have detected the occupational risk of developing respiratory and allergic symptoms in hairdressers exposed to chemicals from bleaching powders and aerosol hair products (66).

It has been shown that vitamin E and selenium reduced radiation-induced transformation in cell culture; the combination was more effective than the individual agents (67, 68). Natural beta-carotene (BC) protected against radiation-induced neoplastic transformation in cell culture (69). Vitamin E and C, and BC reduced radiation-induced mutations and chromosomal damage in mammalian cells in culture (70-78). These studies suggest that free radicals generated during irradiation can induce genetic damage that can be attenuated by antioxidants.

Alpha-lipoic acid, a glutathione-elevating agent, increases the LD₅₀ (a dose producing 50% mortality within 30 days) in mice with a dose reduction factor of 1.26 (79). Vitamin E, Vitamin C and BC protected rodents against the acute effects of irradiation (75, 76, 78, 80-87). Vitamin A and BC protected normal tissue during radiation therapy of cancer in an animal model (88). A combination of vitamin A, C and E protected against radiation-induced myelosuppression during radiation therapy of cancer in an animal model (80). Supplementation with L-selenomethionine and several different types of antioxidants (vitamin C, vitamin E, glutathione, N-acetylcysteine (NAC), alpha-lipoic acid and co-enzyme Q10 and soy bean-derived Bowman-Birk inhibitor) protected human cells in culture and rats in vivo against oxidative stress produced by protons and one GeV (giga-electron volts) iron ions (89-91). The amino acid derivative, L-carnitine has been shown to protect rodents from ionizing radiation-induced cataract formation and brain damage (92, 93). This agent also reduced gamma radiation-induced cochlear damage in guinea pigs (94).

Vitamin A and NAC are thought likely to be effective against radiation-induced carcinogenesis (95). An oral supplementation with alpha-lipoic acid for 28 days lowered the levels of lipid peroxidation among children chronically exposed to low doses of radiation in the area contaminated by the Chernobyl nuclear accident (55). BC supplementation reduced cellular damage in the above population of children (54). A combination of vitamin E and alpha-lipoic acid was more effective than the individual agents (55). These studies in humans demonstrate that very low doses of radiation can increase oxidative stress and induce cellular damage that can be protected by antioxidants. An oral supplementation with BC also protected against radiation-induced mucositis during radiation therapy for the head and neck cancer (84).

Previous animal studies with individual antioxidants have primarily utilized the intraperitoneal route of administration prior to irradiation. Individual antioxidants when administered orally before or after irradiation failed to provide any significant degree of protection. Therefore, a comprehensive combination of multiple dietary and endogenous antioxidants, when administered orally shortly before irradiation, was considered necessary to provide radiation protection. This is especially due to the fact that various antioxidants differ in their mechanisms of action and distribution at cellular and organ levels, while also differing in their internal cellular and organ environments (oxygenation levels, aqueous or lipid components) and their affinity for various types of free radicals. For example, BC is more effective in quenching oxygen radicals than most other antioxidants (96). BC can perform certain biological functions that cannot be produced by its metabolite vitamin A, and vice versa (96, 97). It has been reported that BC treatment enhances the expression of the connexin gene which codes for a gap junction protein in mammalian fibroblasts in culture, whereas vitamin A treatment does not produce such an effect (98). Vitamin A can induce differentiation in certain normal and cancer cells, whereas BC and other carotenoids do not (99, 100). Thus, BC and vitamin A have, in part, different biological functions.

The gradient of oxygen pressure varies within cells. Some antioxidants, such as vitamin E, are more effective as quenchers of free radicals in reduced oxygen pressure, whereas BC and vitamin A are more effective in higher atmospheric pressures (101). Vitamin C is necessary to protect cellular components in aqueous environments, whereas carotenoids and vitamins A and E protect cellular components in lipid environments. Vitamin C also plays an important role in maintaining cellular levels of vitamin E by recycling the vitamin E radical (oxidized) to the reduced (antioxidant) form (102). Also, oxidative DNA damage produced by high levels of vitamin C could be protected by vitamin E. Oxidized forms of vitamin C and vitamin E can also act as radicals; therefore excessive amounts of any of these forms, when used as single agents, could be harmful over a long period of time.

The form of vitamin E used is also important in any clinical trial. It has been established that d-alpha-tocopheryl succinate (alpha-TS) is the most effective form of vitamin both in vitro and in vivo (103, 104). This form of vitamin E is more soluble than alpha-tocopherol and enters cells more readily. Therefore, it is expected to cross the blood-brain barrier in greater amounts than alpha-tocopherol. An oral ingestion of alpha-TS (800 I.U./day) in humans increased plasma levels of not only alpha-tocopherol, but also alpha-TS, suggesting that a portion of alpha-TS can be absorbed from the intestinal tract before hydrolysis (105). This observation is important because the conventional assumption based on rodent studies has been that esterified forms of vitamin E such as alpha-TS, alpha-tocopheryl nicotinate or alpha-tocopheryl acetate, can be absorbed from the intestinal tract only after they are hydrolyzed to alpha-tocopherol. This assumption may not be true for the absorption of alpha-TS in humans.

Glutathione is effective in catabolizing H₂O₂ and anions. However, an oral supplementation with glutathione failed to significantly increase plasma levels of glutathione in human subjects (106), suggesting that this tripeptide is completely hydrolyzed in the G.I. tract. Therefore, NAC and alpha-lipoic acid that increase the cellular levels of glutathione by different mechanisms are utilized in the BioShield formulations.

Other endogenous antioxidants, such as coenzyme Q₁₀, may also have potential value in radiation protection. Since mitochondrial dysfunction may be associated acute and late effects of radiation, and since coenzyme Q₁₀ is needed for the generation of ATP by mitochondria, it is essential to add this antioxidant in multiple micronutrient preparations in order to improve the function of mitochondria. Ubiquinol (coenzyme Q₁₀) scavenges peroxy radicals faster than alpha-tocopherol (107), and like vitamin C, can regenerate vitamin E in a redox cycle (108). Selenium is a co-factor of glutathione peroxidase, and Se-glutathione peroxidase increases the intracellular level of glutathione, a powerful antioxidant. Therefore, selenium and co-enzyme Q10 are included in the BioShield formulations with the intent to achieve optimal protection.

Short-lived free radicals are generated during irradiation, and long-lived free radicals exist after irradiation. In addition, reactive oxygen species are released from inflammatory reactions that occur after irradiation. The inflammatory reactions also release toxic chemicals such as pro-inflammatory cytokines, adhesion molecules and complement proteins all of which are toxic to cells. In addition to free radicals that are generated during irradiation, these post-irradiation biological events should be attenuated in order to maximize the efficacy of potential protective agents. Therefore, pre- and post-exposure supplementation with a protective agent is essential. Antioxidants not only neutralize free radicals, but also reduce inflammatory reactions (109, 110). Thus, several mechanisms in different time frames are involved to maximize effectiveness.

Analysis of the inventors' research, published literature and extensive investigations of the designed multiple antioxidant micronutrients led to the development of the BioShield formulations. This was further based on the demonstration that the proper comprehensive antioxidant combination can neutralize free radicals and reduce the inflammation that contributes to the progression of damage. BioShield is generally regarded as safe and intended to reduce the adverse health risks associated with hazardous exposures in humans. The following scientific data on the efficacy of this formulation for radiation protection was shown in multiple in vivo models including sheep, rabbits, mice and fruit flies.

Sheep appear to be more sensitive to the GI syndrome than are rodents or humans. A pilot study was performed to evaluate the effects of the micronutrient “backbone” of BioShield, a combination of multiple dietary antioxidants, including vitamin A, vitamin C, and vitamin E (d-alpha tocopheryl succinate and d-alpha tocopheryl acetate), selenomethionine, and endogenous antioxidants, including NAC, R-alpha-lipoic acid, and coenzyme Q10. The results showed that a dose of 4.41 Gy produced a GI syndrome associated with a CNS syndrome, causing 100% lethality in seven days in the placebo-fed group. However, an oral administration of the antioxidant mixture daily for a week before and daily for a week after irradiation increased the survival time of irradiated sheep from seven to 38 days. This is a first demonstration in which oral consumption of a comprehensive antioxidant combination before and after irradiation increased the survival time of irradiated animals exhibiting a GI syndrome by about 5-fold.

Rabbits appear to be more sensitive to the GI syndrome than are sheep. A pilot study was performed to evaluate the effects of the same multiple antioxidant micronutrient combination as was used in the sheep. The results showed that a dose of 9.011 Gy produced a GI syndrome associated with a CNS syndrome. About 25% of the irradiated rabbits died of the CNS syndrome within 4 hours and the remaining rabbits died within 7 days. This was expected because the very high exposure dose produced full-blown GI and CNS syndromes. However, the necropsy of those irradiated rabbits dying of CNS syndrome showed that the extensive lung damage noted in the placebo-fed group was virtually eliminated in the BioShield-fed rabbits (FIG. 3).

The autopsy of irradiated rabbits receiving placebo revealed that the lung was necrotic and without lobular architecture (FIG. 3A), whereas the BioShield-fed irradiated rabbits showed minimal pulmonary hemorrhage while maintaining the lobular architecture of the lung (FIG. 3B). This level of protection has not been previously achieved by any orally ingested pharmacological or biological agents.

A pilot study was performed to evaluate the effects of the same multiple antioxidant micronutrient combination as was used in the sheep. The results showed that a radiation dose of 8.5 Gy produced a bone marrow syndrome with 100% lethality within 30 days in the placebo group. Conversely, oral administration of the BioShield mixture daily for 7 days or 24 hours before whole-body gamma-irradiation increased the survival rate of irradiated animals from 0% to 40% (Table 5). Of note, certain polysaccharides when administered intraperitoneally shortly before whole-body irradiation have been reported to be of some radioprotective value in irradiated mice (111-113). However, this level of protection has not been achieved by the oral administration of a single antioxidant or its derivatives before whole-body gamma-irradiation with a dose that produced a bone marrow syndrome dose and 100% mortality.

A pilot study was performed to evaluate the effects on proton radiation-induced cancer of the same multiple antioxidant micronutrient combination as was used in the sheep. The female fruit flies being used were carrying a mutant HOP (TUM-1) gene and become very sensitive to develop leukemia-like cancer when exposed to the radiation. The placebo-fed flies all developed the leukemic lesions. Dietary supplementation with the BioShield mixture for seven days before and during the entire observation period after irradiation prevented the proton radiation-induced cancer. This is the first demonstration in which a genetically-based oncologic disease can be prevented by a unique combination of antioxidant micronutrents. These studies further support the concept of a biological protection strategy against radiation damage as a rational approach. They also suggest that this protection may be effectively achieved after oral administration of a comprehensive combination of antioxidant micronutrients.

The BioShield formulations were developed from an analysis of scientific studies based on the inventors' personal investigations and observations and synthesized on their understanding and application of antioxidant science. The body of work demonstrating the potential efficacy of antioxidant micronutrients is derived from involving at least 12 cell culture experiments, 15 animal studies and five pilot human trials. The uniqueness of the BioShield strategy is that it provides an orally-administered, comprehensive mixture of multiple dietary and endogenous antioxidants including glutathione-elevating agents, the final formulation of which has been extensively evaluated. Previous attempts at developing protective agents have usually depended on the published work of others or on the value of individual (or limited combinations) of antioxidants, many of which have proven to be toxic in humans.

Human peripheral lymphocytes when irradiated with a dose 10 mGy (10 mSv) (equivalent to a dose delivered to patients in a single CT scan) showed an increased number of cells with DNA double-stranded breaks (DSBs) compared to unirradiated control cells (114). The DNA DSBs, if not prevented or repaired, are very significant inducers of mutagenesis or carcinogenesis. Using the BioShield E1 preparation of multiple dietary and endogenous antioxidants, it was demonstrated in vitro that pretreatment of human peripheral lymphocytes in culture before irradiation with this formulation markedly reduced the number of cells with DNA DSBs. In a separate in vivo experiment, normal individuals were given BioShield E1 orally for a period of 15, 30 and 60 min after which their peripheral blood was drawn. Their lymphocytes were irradiated with the same10 mGy and the number of cells with DNA DSBs was subsequently determined. The results showed that BioShield E1 consumption before irradiation reduced DNA DSBs in two separate assays by about 58% to 63% in these subjects (159). Direct protection of human DNA from radiation effect by an orally-ingested product has never before been demonstrated.

Dietary antioxidants (vitamins A, C and E, BC and selenium) and endogenous antioxidants and glutathione-elevating agents (N-acetyl cysteine, alpha-lipoic acid, coenzyme Q10, and L-carnitine) have been consumed by humans for decades without any reported toxicity. The micronutrients in BioShield are generally regarded as safe and no adverse effects have been seen in animal studies, human trials or current usage by the public (115). Nevertheless, whenever these ingredients are included in a formulation, appropriate information or statement for referral to a health care professional for product approval will be provided. Therefore, at the doses recommended and for consumption only by specific high risk populations, the safety profile may be acceptable because of a generally favorable risk-benefit ratio.

TABLE 1
Summary of estimated effective dose from
average diagnostic radiation procedures

| Type of examination | Effective dose (mSV) |
|---|---|
| Standard radiography | 0.01 to 10.0 |
| Computed tomography | 2.0 to 20.0 |
| Nuclear medicine | 0.3 to 20.0 |
| Interventional procedure | 5.0 to 70.0 |

Data taken from Reference (28).

TABLE 2
Estimated doses of ionizing radiation
delivered during specific events

| Procedure type | Effective dose (mSv) |
|---|---|
| Chest or dental x-ray | 0.01 |
| Electron-beam CT (cardiac) | 1.0-1.3 |
| Electron-beam CT coronary angiography | 1.5-2.0 |
| Catheter coronary angiography | 2.1-2.5 |
| Electron-beam CT whole body | 5.2 |
| CT (head) | 2.0 |
| CT (abdomen) | 10.0 |
| Barium enema | 7.0 |
| Upper G.I. Series | 3.0 |
| I.V. Urogram | 2.5 |
| Lumbar spine | 1.3 |
| Mammogram | 7.0 |
| Passenger flight Athens to New York | 0.06 |
| Occupational annual dose limit | 50.0 |
| General public annual dose limit | 1.0 |
| Background annual dose at sea level | 1.0 |

Occupational and general public dose limit does not include background radiation. Data taken from Reference (111).

TABLE 3
Estimated doses of radiation from selected radioactive nuclides
administered once during nuclear medicine procedures

| Procedure type | Effective dose (mSv) |
|---|---|
| 18F-Flurodeoxyglucose, 10 mCi | 4.8 |
| 99mTc-MAA lung scan, 5 mCi | 0.60 |
| 99mTc-HDP bone scan, 20 mCi | 4.0 |
| 201Tl Thallium scan, 3 mCi | 0.60 |

Data taken from Reference (111).

TABLE 4
Estimated increase in cancer risk from exposure
to one computed tomography (CT) scan

| Source and dose of | Increase in cancer |
|---|---|
| radiation individuals | risk in exposed |
| One CT scan | 1 per 1000 (10, 17, 18) |
| | 1 per 1200 (19) |
| | 2.9 per 1000 (12) |
| | 0.26 per 1000 female (20) |
| | 0.2 per 1000 male (20) |
| 15 mSv | 1 per 750 (27) |
| 20 mSV | 1 per 500 (27) |
| 25 mSv | 1 per 400 (27) |

Data were taken from reference numbers in parentheses above.

TABLE 5
Effect on survival in mice of antioxidants or placebo administered
orally at a dose of 222.5 mg/kg of body weight 7 days before
exposure to various doses of whole-body gamma-irradiation.

| Agents | Dose (Gy) | % survival |
|---|---|---|
| Placebo | 7.5 Gy | 90 |
| Antioxidant mixture | 7.5 Gy | 80 |
| Placebo | 8 Gy | 30 |
| Antioxidant mixture | 8 Gy | 60 |
| Placebo | 8.5 Gy | 0 |
| Antioxidant Mixture | 8.5 Gy | 40 |
| Placebo | 9.0 Gy | 0 |
| Antioxidant mixture | 9.0 Gy | 0 |
| Placebo | 9.25 Gy | 0 |
| Antioxidant mixture | 9.25 Gy | 0 |

Each group contained 10 animals.

Ionizing radiation (X-rays and gamma rays) has proven to be a double-edged sword in clinical Medicine since its discovery by Dr. Wilhelm Roentgen in 1895 (1,2). Energy wavelength progresses along the electromagnetic continuum from longer ranges (radiowaves, microwaves, infrared, and heat waves) to medium wavelengths (visible light, ultraviolet light) to shorter wave lengths (ionizing radiation, e.g., x-rays and gamma rays). It is these x-rays and gamma rays that are able to drive electrons out of their normal atomic orbits with enough kinetic energy to generate charged molecules (including free radicals) that damage cells. In addition to the initial realization by the medical community that ionizing radiation could detect as well as treat human diseases, came the unfortunate demonstration that it could also induce serious illness.

In fact, most of the ionizing radiation to which the human population is exposed, other than that received from environmental sources, is from the diagnostic and screening imaging machines employed by today's clinical healthcare professionals. For example, in the past, x-ray-induced skin cancers were noted with higher frequency in radiologists. Obviously, whenever x-rays are employed, it is done with caution so that patients and healthcare providers are exposed to as low a does as possible. Physicists and nuclear engineers have devised improved equipment and radiation beam delivery systems to reduce the level of diagnostic radiation dose without compromising the quality of images. However radiation biologists agree that there is no threshold dose below which there is no risk of cellular damage. In fact, even a single radiation track that crosses a cellular nucleus has a very low, but finite, probability of generating damage that may result in cellular dysfunction, somatic and heritable mutations, and subsequent genetic implications.

While most clinical radiologists believe the risks of x-ray exposure are very small, residual biologic effects from alteration in structure are dependent on whether the cell repairs its injured components. Although the vast majority of damage is repaired, some may be unrepaired or misrepaired and there in lies the problem. In adults, most radiation researchers consider cancer induction to be the most important somatic effect of low dose ionizing radiation and this outcome may occur in nearly all the tissues of the human body. Academic radiologists are also raising future disease concerns regarding pediatric age groups because of the increased numbers of imaging studies now being performed in younger populations (3). In light of these concepts the healthcare profession states that ionizing radiation exposure should only occur when there is a defined healthcare benefit, or indicated when the risk-benefit ratio is favorable to the patient. The critical concept has been always to protect humans by physical local factors, such as shielding and decreasing doses and x-rays times. However, no one has previously considered the additional aspects to a strategy of systemic biological protection.

Recent advances in imaging technology have made possible the detection of many illnesses such as heart disease, cancer, neurologic diseases, arthritis and other acute or chronic conditions. It is also significant development that this technology may detect the problem at an early stage when treatment interventions allow for less invasive therapeutic procedures and/or surgical operations and yet achieve improved health outcomes. In this environment, the number of diagnostic x-rays performed is truly enormous. It was estimated in the United States for the period 1985 to 1990 at least 800 diagnostic studies per 1,000 population were performed and this excluded dental x-rays and nuclear medicine (4). The importance of these finding can be appreciated since it is probable that frequent low dose radiation exposures may be more damaging than a single higher dose exposure on the criteria of gene mutations and cancer promotion.

The current era has seen an explosion of diagnostic imaging equipment including the introduction of computed tomography, digital radiography, expanded nuclear medicine applications, interventional radiology, and lengthening fluoroscopic procedures. In concert with these technical innovations, the concept of early disease detection and screening large populations to employ illness prevention strategies will generate further rapid expansion of members of imaging studies with increased ionizing radiation exposure to the public. As a direct consequence of this new proactive healthcare approach, imaging will be performed in many more, otherwise healthy, people and asymptomatic “at risk” populations. In addition, initial exposures will occur at an earlier age and the mandate of serial follow-up imaging will result in an overall greater frequency of x-ray studies.

The doses of ionizing radiation exposure in imaging studies vary dramatically from less than 0.1 rem (1 millisievert, mSv, for x-rays and gamma rays, 1 rem=1 rad) per test for some procedures to others that involve levels in some organs in excess of 10 rem per test. Table 1 lists a sampling of common studies (5-8). Note that while the red marrow dose is usually the reported “standard”, the actual target organ dose is actually often significantly higher. For example, mammography exposes the actual breast tissue to approximately 700 mrem, virtually equal to the total skin entrance dose. Likewise, thallium scanning exposes the thorax to approximately 1000 mrem, about 20 times the red marrow dose.

TABLE 6

| Procedure | Effective Dose | Equivalent (HE) | Skin Entrance Dose |
|---|---|---|---|
| Diagnostic X-ray | mSv* | mrem | mrem |
| Chest AP, 100 kVp | 0.015 | 1.5 | 10 |
| Lumbar spine AP, 80 kVp | 0.273 | 27.3 | 359 |
| Upper G.I. | 4.1 | 410 | 2300/min |
| Coronary angioplasty | 50-150/min | 5000-15000/min | 25000/min |
| Head CT | 0.8-5 | 80-500 | 4500 |
| Abdomen CT | 6-24 | 600-2400 | 2000 |
| Dental | 0.01 | 1 | 350 |
| Electron beam CT heart | 0.14-0.3 | 14-30 | 150 |
| Mammogram | ** | ** | 700 |
| Nuclear medicine | mSv | mrem | mrem |
| 18F-Fluorodeoxyglucose, | 9.99 | 999 | NA |
| 10 mCi | | | |
| 99mTc-MAA Lung Scan | 2.03 | 203 | NA |
| (perfusion only) 5 mCI | | | |
| 99mTc-HDP Bone scan 20 mCi | 5.92 | 592 | NA |
| 201TI Thallium scan 3 mCi | 25.53 | 2553 | NA |

*Seivert is the official unit of biological radiation dose.
One Sv = 100 rem.
ND = Data not available
** = Dose negligible
NA = Not Applicable

Depending on the age of the individual, frequency of testing, exposure time, and total dose, the diagnostic or screening imaging studies could increase the risk of somatic damage (some forms of cancer such as leukemia, breast, and thyroid) as well as genetic damage (such as with gonadal exposure) in the target population. In fact, radiation experts are beginning to call for special attention to issues of exposure from CT Scanning in young patients (9). It should be emphasized that the risk of radiation injury produced by diagnostic doses below 0.5 rem is very small in comparison to other agents that are present in the diet of the natural environment. However, regardless of the “insignificant” risk with any individual exposure or imaging event, the total effects of ionizing radiation are on-going, cumulative over time, have the potential for lifelong expression, and may have a future generational genetic impact.

It should be anticipated that as more sophisticated imaging studies are available for diagnosis and screening, the individual small risks may add up over a lifetime. For example, nuclear medicine has been expanded to new techniques which include intravenous systemic injection of radionuclides and expose various body organs to differing radiation doses (10). The recent impact of interventional techniques often combined with surgical procedures also increases the imaging risks. Furthermore, advance fluoroscopic imaging used for technical procedures such as percuataneous transluminal angioplasty, transhepatic cholangiography, stent and drainage placements as well as venous access procedures may involve significant radiation exposure (11). In fact by the year 2000 in the United States alone, about 750,000 patients underwent coronary balloon angioplasty (12). Finally, the most recent technical innovations utilized in screening procedures, such as spiral and electron beam computed tomography for heart, lung, colon, and total body scanning are new clinical areas where issues of radiation dosimetry have to be considered (13,14).

Currently, the FAA and airlines consider flight personnel (including flight attendants) as radiation workers. As such, they are allowed a regulatory dose limit 50 times the dose limit allowable to the general public. According to recent estimates, over 400,000 frequent fliers travel over 75,000 air miles each year, which means that they will exceed radiation dose limits to the general public from galactic (cosmic) radiation during flight (15). The radiation exposure during flight varies with altitude, flight time, air route, and solar flare activity. As an example, a routine flight form New York to Chicago (highest altitude 37,000 feet) yields a radiation dose rate of 0.0039 mSv per block hour. (The block hour begins when the aircraft leaves the blocks before takeoff and ends when it reaches the blocks after landing.) A flight from Athens, Greece to New York (highest altitude 41,000 feet) yields a radiation dose rate of 0.0063 mSv per block hour. The total radiation dose from the New York to Chicago route is 0.0089 mSv and the Athens t New York flight is 0.0615 mSv. For reference, the annual exposure limit for the general public is 1 mSv. The only remediation recommended by the FAA for radiation exposure during fight to is to limit flight and avoid traveling during periods of increased solar flare activity. Airline crew members flying long-haul high-altitude routes receives, on average, greater exposure each year than do radiation workers in ground-based industries where radioactive sources or radiation-producing machines are used (16).

The United States military is aware of and concerned about potential radiation exposures to out troops. Perhaps the most obvious population risk in the military is pilots flying long, high-altitude missions. The expected radiation doses would be in accordance with the estimates outlined above. The most recent U.S. Army study on the issue recognizes four nuclear radiation exposure risk categories of military personnel based on their likelihood and extent of exposure (17, Table 2). The army currently has three radiation protection programs to address these risk categories. One is applied to those individuals whose duties parallel those of civilian radiation workers. These include military personnel, such as x-ray technicians, radiologists who do radiological examinations, researchers who use radionuclides, and technicians who maintain radioactive commodities, such as radiation detection instruments and calibration sources. The second applies to soldiers whose primary occupation does not usually expose them to radiation. These are soldiers who might respond to a military situation, such as that covered by Allied Command Europe Directive (ACE) 80-63, in which radiation is present, but at doses not exceeding 700 mSv. The third category applies to those situations involving extremely high radiation exposure, such as nuclear war.

TABLE 7
Revised, Low-Level Radiation Guidance for Military Operations

| Total | | Radiation | | Increased Risk |
|---|---|---|---|---|
| Cumulative | | Exposure State | Recommended | of Long Term |
| Dose* | | Category | Actions | Fatal Cancer** |
| <0.5 | mGy | 0 | None | None |
| 0.5-5 | mGy | 1A | Record individual | 1:4,000 |
| | | | dose readings | |
| | | | Initiate periodic monitoring | |
| 5-50 | mGy | 1B | Record individual | 1:400 |
| | | | dose readings | |
| | | | Continue monitoring | |
| | | | Initiate rad survey | |
| | | | Prioritize tasks | |
| | | | Establish dose control measure as | |
| | | | part of operations | |
| 50-100 | mGy | 1C | Record individual | 1:200 |
| | | | dose readings | |
| | | | Continue monitoring | |
| | | | Update survey | |
| | | | Continue dose-control measures | |
| | | | Execute priority tasks only*** | |
| 100-250 | mGy | 1D | Record individual | 1:80 |
| | | | dose readings | |
| | | | Continue monitoring | |
| | | | Update survey | |
| | | | Continue dose control measures | |
| | | | Execute critical tasks only**** | |
| 250-700 | mGy | 1E | Record individual | 1:30 |

dose readings
Continue monitoring
Update survey
Continue dose control measures
Execute critical tasks only

*The use of the measurement millisievert is preferred in all cased. However, due to the fact that normally the military has only the capability to measure milligray (mGy), as long as the ability to obtain measurement in millisievert is not possible, U.S. forces will use milligray. For whole body gamma irradiation, 1 mGy is equal to 1 mSv. All doses should be kept as low as reasonably achievable (ALARA). This will reduce the risk to individual soldiers and will retain maximum operational flexibility fur future employment of exposed soldiers.
**This is in addition to the 1:5 and 1:4 incidence of fatal cancer among the general population. Increased risk is given of induction of fatal cancer (losing an average of 24 years of life for personnel ages 20-30 years). It must be noted that higher radiation dose rates produce proportionately more health risks than the same total dose given over a longer period.
***Examples of priority tasks are those missions to avert danger to persons or to prevent damage from spreading.
****Examples of critical tasks are those missions required to save lives.

This study committee made four recommendations:

1) When making decisions, commanders should consider the long-term health effects that any action may have on their troops. This recommendation was extended such that it became standard operating policy.

2) The U.S. Department of Defense should develop and clearly express an underlying philosophy for radiation protection. Specifically, the committee suggested,
a. application and adaptation of the system recommended by the International Commission of Radiological Protection,
b. in peacetime or nonemergency situations, soldiers should be accorded the same level of protection accorded civilians, and
c. in settings in which an intervention is required and specific numerical dose limits are neither applicable nor practical, commanders should justify the mission (there is more benefit than risk), examine competing risks, and optimize the mission (identify way to minimize dose without jeopardizing the mission).

3) Military personnel should receive appropriate training in both radiation effects and protection. Their training will need to vary on the basis of the particular level of potential exposure and the task at hand.

4) A program of measurement, recording, maintenance, and use of dosimetry and exposure information is essential.

The programs, once again, include no protection measures other than controlling time, distance, and physical shield.

Radiation workers experience a broad spectrum of working conditions that have radiation exposure as a normal part of the workplace environment. Examples include medical radiology workers, nuclear power plant workers, and worker who use radiation and radioactive materials in research. As mentioned above, commercial flight crews are also considered to be radiation workers. Owing to this occupational classification, radiation workers are allowed to receive 50 times the radiation dose allowed to the general public. Radiation workers also differ from the general public in that they receive training about the risks of radiation exposure and are monitored for their radiation exposure as part of their working paradigm. The nuclear regulatory commission (NRC) has established occupational dose limits as noted previously and procedures for monitoring and record-keeping. These standards and regulations rely solely on time, distance, and physical shielding as methods of radiation protection.

### SUMMARY OF THE INVENTION

The increasing use of x-ray-based equipment in medical and dental diagnosis has raised concerns about the potential hazards of such procedures in increasing the risk of cancer and somatic and heritable mutations. These risks also exist in radiation workers in several industries (including frequent flyers) and those in government and military occupations who are also exposed to higher doses of radiation per year than non-radiation workers. In addition, the constant use of cellular phone technology and the occurrence of natural disasters have further impacted the hazards associated with a broad spectrum of other environmental exposures that people deal with on a daily basis. Finally, the increasing hostilities in the world can lead to terrorist activities that may unleash a host of potentially dangerous agents.

Much of the adverse health effect from environmental hazards is related to excess free radical-induce oxidative damage, inflammation and reduced immune function. It is well established by the studies summarized in this application and the supporting scientific references cited that an antioxidant micronutrient strategy can neutralize free radicals, decrease inflammation and enhance immune function. Therefore, supplementation with the BioShield formulations can provide biological protection against a spectrum of adverse exposures and provide science- and evidence-based approaches for this effect.

A specific subset of these exposures relates to radiation and the biological strategy described herein adds a significant incremental body, organ, tissue and cellular protection above that provided by physical means alone. Since radiation exposure generates excessive amounts of free radicals and pro-inflammatory cytokines, and since antioxidants neutralize free radicals and reduce the levels of pro-inflammatory cytokines, the BioShield formulations provide a rational protective approach (11). The consumption of the proper, comprehensive antioxidant micronutrient combination would extend the concept of ALARA from radiation dose to biological protection. The present invention highlights this novel concept, supported by extensive data, and can be referred to as PAMARA (protection as maximum as reasonably achievable) based on the BioShield platform of micronutrient supplementation.

In one embodiment of the invention a formulation consisting essentially of antioxidants, the antioxidants are selected from the group consisting essentially of Vitamin A, Vitamin C, Vitamin E, L-cysteine, N-acetylcysteine, R-alpha-lipoic acid, Selenium, Natural mixed carotenoids and mixtures thereof.

In a further embodiment, the formulation comprises at least one glutathione elevating agent.

In yet another embodiment, the a dosage level of antioxidants for the formulation is proportionate to the radiation level exposed to by a human.

In yet a further embodiment, the formulation is designed for a human who exposed to an effective dose of ionizing radiation of 0.5 mSv or less, radon, smoking, biological agents, or radiation.

In a further embodiment, the formulations is for health care, aviation, military, and government employees.

In still another embodiment for the formulation, the antioxidants consist essentially of 5,000 IU of Vitamin A, 500 mg of vitamin C, 400 international units of d-alpha tocopheryl acid succinate, 200 mg of L-cyteine, 30 mg of R-alpha-lipoic acid, and 250 mg of N-acetyl cysteine, 100 mcg of Selenium, and 15 mg of natural mixed carotenoids.

In yet another embodiment, formulation is designed for a human who is consistently exposed to an effective dose of ionizing radiation of 0.5-5 mSv, radon, smoking, occupational chemicals, pesticides, cellular phone technology, air pollution and biological agents.

In still a further embodiment, the antioxidants consist essentially of 3,00 IU of Vitamin A, 1,000 mg of buffered vitamin C, 400 international units of d-alpha tocopheryl acid succinate, 200 mg of L-cysteine, 250 mg of N-acetyl cysteine, 800 IU of Vitamin D, 4 mg of Vitamin B1, 5 mg of Vitamin B2, 30 mg of Vitamin B3, 5 mg of Vitamin B6, 800 mcg of Folic acid, 10 mcg of Vitamin B12, 200 mcg of Biotin, 10 mg or Pantothenic acid, 250 mg calcium, 125 mg of Magnesium, 15 mg of Zinc, 200 mcg of Selenium, 50 mcg of Chromium, 30 mg R-alpha-lipoic acid, 100 mg of L-carnitine, and 15 mg of natural mixed carotenoids.

In still a another embodiment, the formulation is designed for a human who receives an effective dose of ionizing radiation of 5-250 mSv, nuclear accidents, terror attacks, or potentially lethal doses of radiation.

In another embodiment, the antioxidants consist essentially of 3,00 IU of Vitamin A, 1,000 mg of buffered vitamin C, 400 international units of d-alpha tocopheryl acid succinate, 200 mg of L-cysteine, 250 mg of N-acetyl cysteine, 800 IU of Vitamin D, 4 mg of Vitamin B1, 5 mg of Vitamin B2, 30 mg of Vitamin B3, 5 mg of Vitamin B6, 800 mcg of Folic acid, 10 mcg of Vitamin B12, 200 mcg of Biotin, 10 mg or Pantothenic acid, 250 mg calcium, 125 mg of Magnesium, 15 mg of Zinc, 200 mcg of Selenium, 50 mcg of Chromium, 30 mg R-alpha-lipoic acid, 100 mg of L-carnitine, and 15 mg of natural mixed carotenoids.

In yet another embodiment, antioxidants consist essentially of:

| | | |
|---|---|---|
| Vitamin A (retinyl palmitate, fish oil or natural mixed carotenoids) | 500-5,000 | IU |
| Vitamin C (calcium ascorbate, citrus, rose or berry products) | 50-500 | mg |
| Vitamin E (d-alpha tocopheryl succinate and/or d-alpha tocopherol, | 50-400 | IU |
| vegetable or wheat germ products or natural tocopheryl) | | |
| L-cysteine (or natural food sources-e.g. soy extracts, sesame or flaxseeds, | 0.01-300 | mg |
| oat bran, wheat germ, Swiss cheese, peas or whole grain extracts) | | |
| N-acetylcysteine | 0.01-250 | mg |
| R-alpha-lipoic acid (R+, R−, yeast or liver sources) | 5-60 | mg |
| Selenium (L-selenomethionine, sodium selenite/selenate or natural forms) | 25-300 | mcg |
| Natural mixed carotenoids (sea, algal, plant or other natural sources) | 0.01-45 | mg |

In still another embodiment, the formulation consisting essentially of Vitamin D, Vitamin B1, Vitamin B2, Vitamin B3, Vitamin B6, Folic Acid, Vitamin B12, Biotin, Pantothenic acid, Calcium, Magnesium, Zinc, Selenium, Chromium, Coenzyme Q10, L-carnetine, and mixtures thereof.

In yet another embodiment, the antioxidants consist essentially of:

| | | |
|---|---|---|
| Vitamin D (cholecalciferol-D3 or fish oils) | 100-4000 | IU |
| Vitamin B1 (thiamine mononitrate or yeast sources) | 1-10 | mg |
| Vitamin B2 (riboflavin or yeast sources) | 1-20 | mg |
| Vitamin B3 (niacinamide, nicotinic acid, nicotinamide or yeast sources) | 1-100 | mg |
| Vitamin B6 (pyridoxine or yeast sources) | 1-50 | mg |
| Folic acid (folate, yeast or liver source) | 100-2,000 | mcg |
| Vitamin B12 (cyanocobalamin, methylcobalamin or yeast sources) | 1-50 | mcg |
| Biotin (d-biotin or liver sources) | 50-600 | mcg |
| Pantothenic acid (d-calcium pantothenate, rice bran, yeast or liver sources) | 1-50 | mg |
| Calcium (citrate, ascorbate, lactate or plant based sources) | 50-1,000 | mg |
| Magnesium (citrate, lactate, gluconate, oxide, carbonate, hydroxide, chloride, sulphate, | 25-500 | mg |
| lactic acid, Jay phosphate, tribasic phosphate or natural forms) | | |
| Zinc (glycinate, gluconate, oxide, sulphate or natural forms) | 2-45 | mg |
| Selenium (L-selenomethionine, sodium selenite/selenate or natural forms) | 25-300 | mcg |
| Chromium (picolinate, chromic chloride or natural forms) | 10-200 | mcg |
| Coenzyme Q10 (ubiquinone, organ meat or natural ingredient sources-e.g. | 0.01-100 | mg |
| Agrobacterium tumefaciens, Paracoccus denitrificans or Pseudomonas aeruginosa) | | |
| L-carnitine (fumarate or natural forms) | 20-400 | mg |

In yet another embodiment, a method of manufacturing the formulation, a method comprising admixing antioxidants, the antioxidants consist essentially of:

| | | |
|---|---|---|
| Vitamin A (retinyl palmitate, fish oil or natural mixed carotenoids) | 500-5,000 | IU |
| Vitamin C (calcium ascorbate, citrus, rose or berry products) | 50-500 | mg |
| Vitamin E (d-alpha tocopheryl succinate and/or d-alpha tocopherol, | 50-400 | IU |
| vegetable or wheat germ products or natural tocopheryl) | | |
| L-cysteine (or natural food sources-e.g. soy extracts, sesame or flaxseeds, | 0.01-300 | mg |
| oat bran, wheat germ, Swiss cheese, peas or whole grain extracts) | | |
| N-acetylcysteine | 0.01-250 | mg |
| R-alpha-lipoic acid (R+, R−, yeast or liver sources) | 5-60 | mg |
| Selenium (L-selenomethionine, sodium selenite/selenate or natural forms) | 25-300 | mcg |
| Natural mixed carotenoids (sea, algal, plant or other natural sources) | 0.01-45 | mg |

In still a further embodiment, a method of manufacturing the formulation a method comprising admixing antioxidants, said antioxidants consist essentially of:

| | | |
|---|---|---|
| Vitamin D (cholecalciferol-D3 or fish oils) | 100-4000 | IU |
| Vitamin B1 (thiamine mononitrate or yeast sources) | 1-10 | mg |
| Vitamin B2 (riboflavin or yeast sources) | 1-20 | mg |
| Vitamin B3 (niacinamide, nicotinic acid, nicotinamide or yeast sources) | 1-100 | mg |
| Vitamin B6 (pyridoxine or yeast sources) | 1-50 | mg |
| Folic acid (folate, yeast or liver source) | 100-2,000 | mcg |
| Vitamin B12 (cyanocobalamin, methylcobalamin or yeast sources) | 1-50 | mcg |
| Biotin (d-biotin or liver sources) | 50-600 | mcg |
| Pantothenic acid (d-calcium pantothenate, rice bran, yeast or liver sources) | 1-50 | mg |
| Calcium (citrate, ascorbate, lactate or plant based sources) | 50-1,000 | mg |
| Magnesium (citrate, lactate, gluconate, oxide, carbonate, hydroxide, chloride, | 25-500 | mg |
| sulphate, lactic acid, Jay phosphate, tribasic phosphate or natural forms) | | |
| Zinc (glycinate, gluconate, oxide, sulphate or natural forms) | 2-45 | mg |
| Selenium (L-selenomethionine, sodium selenite/selenate or natural forms) | 25-300 | mcg |
| Chromium (picolinate, chromic chloride or natural forms) | 10-200 | mcg |
| Coenzyme Q10 (ubiquinone, organ meat or natural ingredient sources-e.g. | 0.01-100 | mg |
| Agrobacterium tumefaciens, Paracoccus denitrificans or Pseudomonas aeruginosa) | | |
| L-carnitine (fumarate or natural forms) | 20-400 | mg |

If it could be possible to devise a strategy to reduce the potential adverse effects of radiation exposure, it certainly seems reasonable that this approach should be undertaken regardless of how small the actual risk of injury might be. Federal law by regulatory code (C.F.R. 21 and C.F.R. 35) emphasizes ALARA guidelines as they relate to occupational radiation exposure. This concept should be extended to the biological consequences of the doses received by all classes of exposed individuals, including patients. The guidelines could be referred to as DALARA (damage as low as reasonably achievable), whereby both the dose and its harmful consequences could be minimized without interfering with the efficacy, ease, or cost of diagnostic procedures, or occupational and other activities. This novel concept, supported by extensive data, is based on reducing radiation-derived free radical damage by antioxidant supplementation. Special attention needs to be given to population groups under chronic risk situations like frequent fliers, radiation workers, flight crews, and military personnel in combat theatres of operation. In such cases, episodic dosing with antioxidants is not adequate to achieve ALARA principles. These population groups should achieve and maintain higher antioxidant loads than person with little or no expectation of radiation exposure.

In accordance with the present invention, twice daily dosing with a properly designed multiple antioxidant formulation is employed to maintain desired antioxidant loads in the body.

When chronically exposed (or chronic risk of exposure) individuals can be reasonably expected to incur an acute exposure, such as dangerous combat missions or any flight operations, they should supplement their regular antioxidant regimen with additional doses of selected antioxidants to protect against the anticipated exposure.

More particularly, the present invention is directed to a method for protecting humans in need of such protection from physical damage caused by ionizing radiation comprising administering to said humans on a defined basis prior to and after exposure to such radiation a plurality of antioxidants at a dosage level directly proportional to the radiation level likely to be encountered.

### BRIEF DESCRIPTION OF THE DRAWINGS

The accompanying drawings are included to provide a further understanding of the present invention. These drawings are incorporated in and constitute a part of this specification, illustrate one or more embodiments of the present invention and together with the description, serve to explain the principles of the present invention.

FIGS. 1A and 1B shows immune function of participants in study taking supplements; and

FIG. 2 is a summary of the average value of MDA levels in plasma and urine as well as TLT in participants n the study.

FIG. 3 Lung damage in rabbits exposed to CNS syndrome-causing radiation dose (9.011Gy).

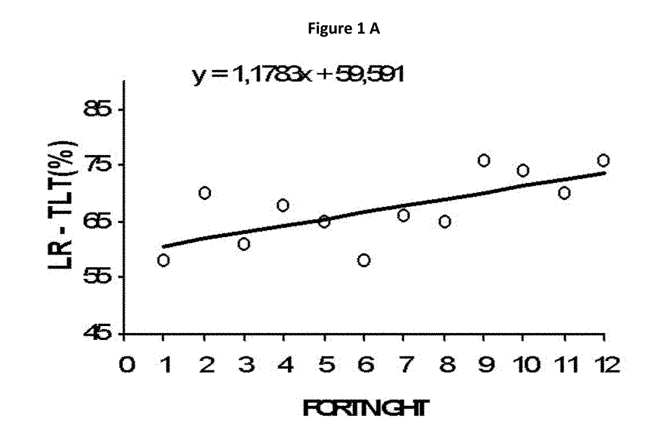

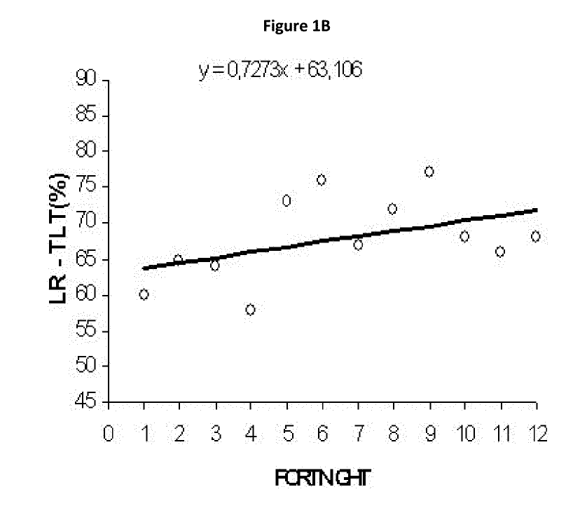

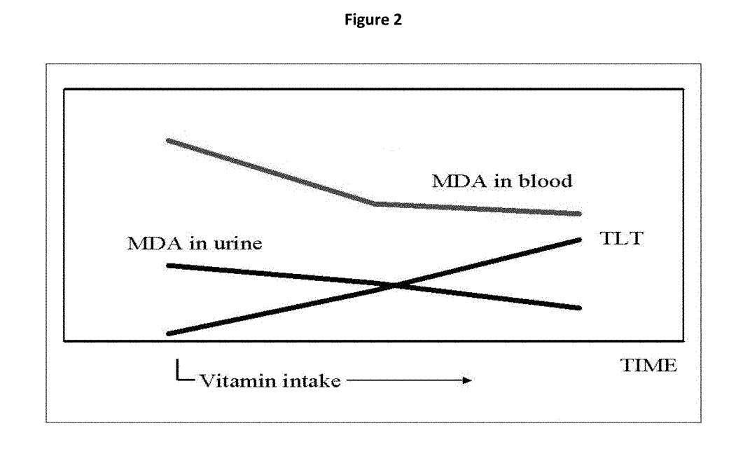

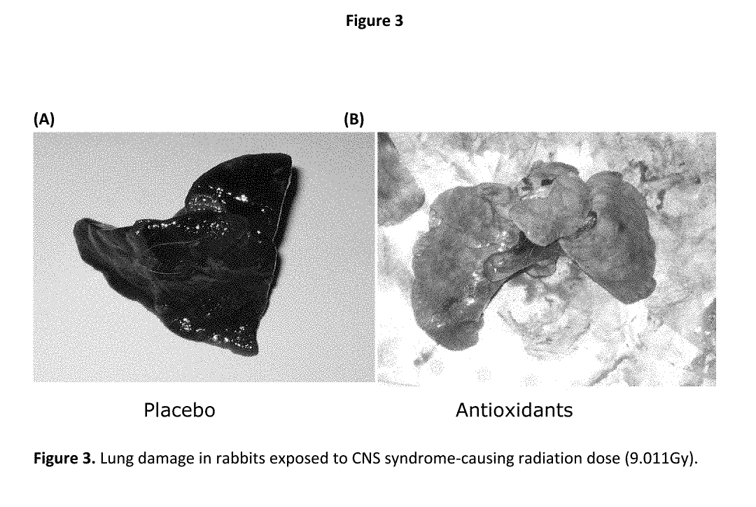

Among those benefits and improvements that have been disclosed, other objects and advantages of this invention will become apparent from the following description taken in conjunction with the accompanying drawings. The drawings constitute a part of this specification and include exemplary embodiments of the present invention and illustrate various objects and features thereof.

### DETAILED DESCRIPTION OF THE INVENTION

As required, detailed embodiments of the present invention are disclosed herein; however, it is to be understood that the disclosed embodiments are merely exemplary of the invention that may be embodied in various forms. The examples disclosed herein are not to be interpreted as limiting, but merely as a basis for the claims and as a representative basis for teaching one skilled in the art to variously employ the present invention.

Although brief medical x-rays themselves may not cause detectable damage, serial imaging, future screening studies (the importance of which cannot be currently predicted), flight exposures, military operations exposures, occupational exposures, and other factors, such as diet, disease status, and environmental exposure, and the like may be clinically significant.

Relevant findings from basic scientific studies underscore this clinical concern. For example, a dose of 2 rem does not cause detectable mutations in normal human lymphocytes in culture. However, if the cells are irradiated with the same dose and treated with caffeine for a few hours after radiation exposure, an increased rate of cellular mutations is observed. This suggests that radiation-induced changes could be repaired in the normal course of events, but that subsequent exposure to caffeine impairs this normal cellular protective mechanism. In addition, a radiation dose that by itself would not be sufficient to induce cancer in an in vitro experimental system is able to do so in the presence of tumor promoters, such as phorbol ester, estrogen, and others. Furthermore, x-rays increase the incidence of cancer in cell culture by several folds when combined with chemical carcinogens, certain DNA viruses, ultraviolet radiation, or ozone exposure. Clearly, the potential hazard of even small radiation doses should not be ignored, since the target population readily interacts with agents present in the diet and environment, as well as other factors present in individual lifestyles.

### Risk Categories

The following risk categories are general guidelines only and refer to acute exposures. The examples listed are not totally inclusive. The actual risk for any particular person may be modified by age and health status. The actual designation for all persons should be determined by healthcare or radiation physics professionals.

Population groups experiencing chronic radiation exposure risk, such as radiation workers (including commercial and military flight crews and field combat personnel), should maintain a higher baseline antioxidant load by taking a multiple antioxidant formulation (SEVAK) two times a day. They should then take the appropriate radioprotective formulation when the acute risk of exposure is expected (daily necessary). Categories 2-4 are equivalent with respect to formulation and can be regarded to be adequate for exposures less than 15 sMv effective dose when used for acute exposures only.

### Category 1: Effective Dose 0.5 mSv or Less

For example: chest x-ray, dental x-ray, abdominal x-ray, skeletal plain films, most commercial flight passengers.

### Category 2: Effective Dose 0.5-5 mSv

For example: diagnose/screening computed tomography, urologic imaging, mammography, flight crews (commercial and military) and other radiation workers.

### Category 3: Internal Radionuclide Exposures

For example: radionuclide imaging.

### Category 4: Effective Dose 5-15 mSv

For example: limited diagnostic fluoroscopy (upper GI series, cholangiography, brain enema).

Category 5: Effective Dose Greater than 15 mSv-250 mSv

For example: prolonged fluoroscopy/interventional radiology (coronary angiography, cerebral angiography, transluminal angioplasty) and some military personnel in combat operations (ground troops and seamen).

### Category 6: Effective Dose 1000-2000 mSv

For example: radiation workers, civilian populations at risk near nuclear reactor sites in the event of an accident, and at risk military personnel in overseas theatres of operation.

Category 7: Effective Dose Greater than 2000 mSv (not Exceeding Bone Marrow Syndrome Doses)

For example: radiation workers, civilian populations at risk near nuclear sites in the event of an accident, and at risk military personnel in overseas theatres of operation.

Hereinafter, the term “imaging study” will be employed to include chest x-ray, dental x-ray, abdominal x-ray, skeletal plain films, diagnostic/screening computed tomography, urologic imaging, mammography, radionuclide imaging, limited diagnostic fluoroscopy, prolonged fluoroscopy/interventional radiology and the like.

The specific example below will enable the invention to be better understood. However, they are given merely by way of guidance and do not imply any limitation.

### Example 1: Baseline Formulation (SEVAK)

| | | |
|---|---|---|
| Vitamin A (palmitate) | 5,000 | I.U. |
| Natural mixed carotenoids | 15 | mg |
| Vitamin D-3 (cholecalciferol) | 400 | I.U. |
| Natural source Vitamin E | | |
| (d-alpha tocopherol) | 100 | I.U. |
| (d-alpha tocopheryl acid succinate) | 100 | I.U. |
| Buffered Vitamin C (calcium ascorbate) | 500 | mg |
| Thiamine mononitrate | 4 | mg |
| Riboflavin | 5 | mg |
| Niacinamide ascorbate | 30 | mg |
| d-calcium pantothenate | 10 | mg |
| Pyridoxine hydrochloride | 5 | mg |
| Cyanocobalamin | 10 | μg |
| Folic acid (Folacin) | 800 | μg |
| D-Biotin | 200 | μg |
| Selenium (1-seleno methionie) | 100 | μg |
| Chromium picolinate | 50 | μg |
| Zinc glycinate | 15 | mg |
| Calcium citrate | 250 | mg |
| Magnesium citrate | 125 | mg |

### Radioprotective Formulations: (Boost Formulations)

### Example 2: For Category 1 Personnel

| | | |
|---|---|---|
| Vitamin C (calcium ascorbate) | 250 | mg |
| Natural source vitamin E | 200 | I.U. |
| (d-alpha tocopheryl acid succinate) | | |
| N-acetyl cysteine | 250 | mg |

Complete dosage to be taken 1 hour prior to an imaging study.

### Example 3: For Category 2 Personnel

| | | |
|---|---|---|
| Vitamin C (calcium ascorbate) | 500 | mg |
| Natural source vitamin E | 400 | I.U. |
| (d-alpha tocopheryl acid succinate) | | |
| N-acetyl cysteine | 250 | mg |
| Natural mixed carotenoids | 15 | mg |
| Alpha lipoic acid | 30 | mg |

Complete dosage to be taken 1 hour prior to an imaging study or prior to each flight.

### Example 4: For Category 3 Personnel

| | | |
|---|---|---|
| Vitamin C (calcium ascorbate) | 500 | mg |
| Natural source vitamin E | 400 | I.U. |
| (d-alpha tocopheryl acid succinate) | | |
| N-acetyl cysteine | 250 | mg |
| Natural mixed carotenoids | 15 | mg |
| Alpha lipoic acid | 30 | mg |

Complete dosage to be taken 1 hour prior to an imaging study and 24 hours and 48 hours after the imaging study.

### Example 5: For Category 4 Personnel

| | | |
|---|---|---|
| Vitamin C (calcium ascorbate) | 500 | mg |
| Natural source vitamin E | 400 | I.U. |
| (d-alpha tocopheryl acid succinate) | | |
| N-acetyl cysteine | 250 | mg |
| Natural mixed carotenoids | 15 | mg |
| Alpha lipoic acid | 30 | mg |

Complete dosage to be taken 24 hours and 1 hour prior to an imaging study 24 hours after the imaging study.

### Example 6: For Category 5 Personnel

| | | |
|---|---|---|
| Vitamin C (calcium ascorbate) | 500 | mg |
| Natural source vitamin E | 400 | I.U. |
| (d-alpha tocopheryl acid succinate) | | |
| N-acetyl cysteine | 500 | mg |
| Natural mixed carotenoids | 30 | mg |
| Alpha lipoic acid | 60 | mg |

Complete dosage to be taken 48 hours, 24 hours and 1 hour prior to an imaging study 24 hours after the imaging study.

### Example 7: For Category 6 Personnel

| | | |
|---|---|---|
| Vitamin C (calcium ascorbate) | 1000 | mg |
| d-alpha tocopheryl acid succinate | 400 | I.U. |
| alpha tocopherol | 200 | I.U. |
| N-acetyl cysteine | 500 | mg |
| Natural mixed carotenoids | 40 | mg |
| Alpha lipoic acid | 100 | mg |

Complete dosage to be taken prior to anticipated exposure or as soon as possible after actual exposure. Continue complete dosage daily for seven days after exposure.

### Example 8: For Category 7 Personnel

| | | |
|---|---|---|
| Vitamin C (calcium ascorbate) | 2000 | mg |
| d-alpha tocopheryl acid succinate | 600 | I.U. |
| alpha tocopherol | 200 | I.U. |
| N-acetyl cysteine | 1000 | mg |
| Natural mixed carotenoids | 50 | mg |
| Alpha lipoic acid | 150 | mg |

Complete dosage to be taken prior to anticipated exposure or as soon as possible after actual exposure. Continue complete dosage daily for seven days after exposure.

It has been estimated that approximately 70-80% of the cellular damage induced by ionizing radiation is caused by free radicals. Therefore, it would be prudent to use agents that would quench these substances formed during x-ray exposure and protect the cells, organs, and total body from such injury.

Since World War II, extensive studies have been undertaken to identify radioprotective compounds that have been shown to be effective when administered before exposure to irradiation. It is important to note that such compounds do not protect cells or organisms if they are administered after the ionizing radiation exposure. For modest radiation dose levels, the protective agents can be absorbed rapidly enough that they could be effective when given immediately before the exposure (within an hour or two). For enough levels of radiation dosage, it might be more desirable to achieve an established steady state of antioxidant concentration in the tissues initially, an then provide a booster dose of radioprotective agent immediately prior to exposure.

Research has determined that sulfhydryl (SH) compounds such as cysteamine, cystamine, and glutathione are among the most important and active intracellular antioxidants. Cysteamine protects animals against bone marrow and gastrointestinal radiation syndromes. The rationale for the importance of SH compounds is further supported by observations in mitotic cells. These are the most sensitive to radiation injury in terms of cell reproductive death and are noted to have the lowest level of SH compounds. Conversely, S-phase cells, which are the most resistant to radiation injury using the same criteria, have demonstrated the highest levels of inherent SH compounds. In addition, when mitotic cells were treated with cysteamine, they became very resistant to radiation. It has also been noted that cysteamine may directly protect cells against induced mutations. Unfortunately, cysteamine is extremely toxic when administered to human beings and, therefore, cannot itself be utilized in a radioprotective antioxidant regimen.

Thus, other SH compounds sharing the same antioxidant characteristics must be considered. Glutathione is a very effective antioxidant. However, when ingested by human beings it is completely hydrolyzed in the intestine and, therefore, can not be used as a radioprotective agent. However, N-acetylcysteine (NAC) and alpha lipoic acid actively increase the intracellular levels of glutathione without causing any toxicity. These rapidly absorbed compounds are tolerated by humans very well and would provide protection against ionizing radiation damage when given prior to the exposure. Indeed, these agents have also been shown to be of radioprotective value in experimental systems. Additional antioxidants such as vitamin E (d-alpha tocopheryl succinate), vitamin C (as calcium ascorbate) and the carotenoids (particularly natural beta-carotene) have been shown to be of marked radioprotective value in animals and in humans. A very recent report by the Armed Forces Radiobiology Research Institute showed good protection by vitamin E against lethal doses of cobalt-60 in mice.

The natural beta-carotene was selected because it most effectively reduces radiation-induced transformation in mammalian cells in culture. The d-alpha tocopheryl succinate form of vitamin E was selected because it is the most effective form of this micronutrient and also actively reduces the incidence of radiation-induced transformation in mammalian cells. This form of vitamin E is a more effective antioxidant than the more commonly utilized alpha tocopherol or other mixtures of tocopherols. Vitamin C as calcium ascorbate is beneficial because it is the most effective nonacidic form available for human use and, therefore, is less likely to cause stomach upset, diarrhea, and other problems that are observed in some individuals when taking therapeutic doses of vitamin C.

The most effective antioxidant approach to the free radical damage related to ionizing radiation-induced injury must utilize multiple micronutrients. It has been determined that multiple antioxidants are more effective than the individual agents themselves, and we propose this approach for several reasons. It is known that vitamin C and vitamin E are synergistic as antioxidants against free radicals because they are able to protect both the aqueous and lipid environments of the cells respectively. Indeed, one study has shown that oral intake of both vitamin C and vitamin E reduces the levels of fecal mutagens formed during digestion more than that produced by either of the individual antioxidants. It also must be recognized that oxygen level may vary widely within the tissues of whole organs or within the individual cells. This is especially true during the biologic insults that may occur with radiation-induced damage. It is known that beta-carotene acts more effectively as an antioxidant in high oxygen pressures, whereas vitamin E is a more effective antioxidant at reduced oxygen pressures.

Finally the body produces several types of free radicals (a myriad of oxygen-derived and nitrogen-derived species) during exposure to ionizing radiation. Clearly, each antioxidant has a different affinity for each specific class of free radicals. In a parallel manner, a combination of antioxidants is more effective in reducing the growth of tumor cells than the individual agents themselves. Therefore, to provide the most effective overall micronutrient approach to protect against radiation injury, a multiple component protocol utilized with a risk-based strategy seems essential and rational.

Most commercially available multiple supplement formulations contain iron, copper, and/or manganese. It is well known that these substances actively generate free radicals when combined with vitamin C. In addition, these minerals are more easily absorbed from the intestinal tract in the presence of antioxidants, such as vitamin C, and thereby increase the body stores of these minerals. Increased iron stores have been associated with many chronic human conditions, including heart disease, cancer and neurological diseases. Therefore, the addition of iron, copper or manganese to any multiple antioxidant preparation has no scientific merit for optimal health or disease prevention. Only in cases where a person has iron-deficiency anemia is a short-term iron supplement essential.

Many commercially available preparations contain heavy metals such as boron, vanadium, zirconium and molybdenum. Sufficient amounts of these metals are obtained from the diet and the daily consumption of excess amounts over long periods of time can be neurotoxic.

Many commercial preparations contain inositol, methionine and choline in varying amounts, e.g., 30 mg to 60 mg. These small doses serve no useful purpose for improving health because 400 mg to 1,000 mg of these nutrients are obtained daily from even the most minimal diet.

Para-aminobenzoic acid (PABA) is present in some multiple vitamin preparations. PABA has no biologic function in mammalian cells and can block the antibacterial effect of sulfonamides. Therefore, the effectiveness of a sulfonamide may be reduced in some patients being treated for bacterial infection.

Commercially sold multiple antioxidant preparations often contain varying amounts of N-acetyl cysteine or alpha lipoic acid. These nutrients are utilized because they are known to increase glutathione levels in cells. Reduced glutathione is a powerful antioxidant and actively protects both normal and cancer cells against radiation damage. Many cancer patients take antioxidant supplements without the knowledge of their oncologists. Therefore, the consumption of antioxidant preparations containing N-acetyl cysteine or alpha lipoic acid by these patients undergoing radiation therapy could interfere with important anti-cancer treatment.

The addition of both natural mixed carotenoids and vitamin A to any multiple vitamin preparation is essential, because beta-carotene not only acts as a precursor of vitamin A, but also performs important biological functions that cannot be performed by vitamin A. Beta-carotene increases the expression of the connexin gene, which codes for a gap junction protein that is necessary for maintaining the normal cellular phenotype. While other carotenoids, such as, lycopene, xanthophylls, and lutein, are also important for health, they can be obtained from an adequate diet with tomato (lycopene), spinach (lutein), and paprika (xanthophylls) in amounts are higher than those that can be supplied from supplements. Therefore, the addition of a few milligrams of lycopene, xanthophylls, and lutein to any multiple vitamin preparation serves no useful purpose for health or disease prevention.

The proper ratio of two forms of vitamin E, d-alpha tocopherol, which is normally present in the body, and d-alpha succinate, to a multiple antioxidant preparation is essential. Alpha tocopheryl succinate is the most effective form of vitamin E inside the cells, where as alpha tocopherol can readily act as an antioxidant in the intestinal tract and in the extracellular environment of the body. Alpha-tocopherol at doses of 20-60 μg/ml can stimulate the immune system, while the beta, gamma, and delta forms at similar doses can inhibit the immune system. This effect of these forms of tocopherol may not be related to their antioxidant action and, since they are less effective than alpha tocopherol, their supplementation is not recommended.

Tocotrienols are also antioxidants, but they may inhibit cholesterol synthesis. Since this activity is not beneficial in healthy individuals, prolonged consumption of tocotrienols as a supplement is not optimal.

Vitamin C is usually administered as ascorbic acid, which can cause stomach upset, diarrhea and other complications in some individuals. However, using the calcium ascorbate form is most suitable because it is non-acidic and has not been shown to produce negative side effects. The use of potassium ascorbate and magnesium ascorbate in any vitamin preparation is unnecessary. Also, any multiple micronutrient preparation should include adequate amounts of B-vitamins (2-3 times of RDA) and appropriate minerals.

The risk of chronic illness may depend upon the relative consumption of protective versus toxic substances. If the daily intake of protective substances is higher than toxic agents, the incidence of chronic illness may be reduced. Since we know very little about the relative levels of toxic and protective substances in any diet, a daily supplement of micronutrients including antioxidants would assure a higher level of preventive protection.

The present invention also provides for the following formulation examples:

### Example 9: Bioshield—R1 in Two Capsules

| | | |
|---|---|---|
| Vitamin C (calcium ascorbate) | 500 | mg |
| d-alpha tocopheryl succinate | 400 | IU |
| Natural mixed carotenoids | 15 | mg |
| Selenomethionine | 100 | mcg |
| n-acetylcysteine | 250 | mg |
| Alpha-lipoic acid | 30 | mg |

### Example 10: Bioshield—R2 in Four Capsules Daily

| Vitamin A (as palmitate)— | 5,000 | |
|---|---|---|
| Vitamin C (as calcium ascorbate) | 1,000 | mg |
| Vitamin E (as d-alpha-tocpheryl succinate) | 200 | IU |
| (as d-alpha-tocopherol) | 200 | IU |
| Vitamin D (as cholocalciferol) | 400 | IU |
| Vitamin B-1 (thiamine mononitrate) | 4 | mg |
| Vitamin B-2 (riboflavin) | 5 | mg |
| Niacin (as niacinamide ascorbate) | 30 | mg |
| Vitamin B-6 (as pyrodioxine HCl) | 5 | mg |
| Folate (Folic acid) | 800 | mcg |
| Vitamin B-12 (as cyanocobalamin) | 10 | mg |
| Biotin | 200 | mcg |
| Pantothenic acid (as d-calcium pantothenate) | 10 | mg |
| Calcium citrate | 250 | mg |
| Magnesium citrate | 125 | mg |
| Zinc (as zinc glycinate) | 15 | mg |
| Selenium (as selenomethionine) | 200 | mcg |
| Chromium (as chromium picolinate) | 50 | mcg |
| Coenzyme Q10 | 30 | mg |
| N-acetylcysteine | 250 | mg |
| Alpha-lipoic acid | 30 | mg |
| Natural mixed carotenoids | 15 | mg |

### Example 11: Bioshield—R3 in Six Capsules/Daily

| Vitamin A (as palmitate)— | 5,000 | |
|---|---|---|
| Vitamin C (as calcium ascorbate) | 1,000 | mg |
| Vitamin E (as d-alpha-tocpheryl succinate) | 400 | IU |
| (as d-alpha-tocopherol) | 200 | IU |
| Vitamin D (as cholocalciferol) | 400 | IU |
| Vitamin B-1 (thiamine mononitrate) | 4 | mg |
| Vitamin B-2 (riboflavin) | 5 | mg |
| Niacin (as niacinamide ascorbate) | 30 | mg |
| Vitamin B-6 (as pyrodioxine HCl) | 5 | mg |
| Folate (Folic acid) | 800 | mcg |
| Vitamin B-12 (as cyanocobalamin) | 10 | mg |
| Biotin | 200 | mcg |
| Pantothenic acid (as d-calcium pantothenate) | 10 | mg |
| Calcium citrate | 250 | mg |
| Magnesium citrate | 125 | mg |
| Zinc (as zinc glycinate) | 15 | mg |
| Selenium (as selenomethionine) | 200 | mcg |
| Chromium (as chromium picolinate) | 50 | mcg |
| Coenzyme Q10 | 30 | mg |
| N-acetylcysteine | 500 | mg |
| Alpha-lipoic acid | 90 | mg |
| Natural mixed carotenoids | 60 | mg |

The formulation of claim 1 BioShield E1 is designed for humans who are or may be episodically exposed to radon, smoking, biological agents, or are workers in health care, military, government or aviation, including frequent flyers. It may also be appropriate for proactive consumers who utilize emergency survivor kits. In addition, the formulation has relevance for people receiving chest x-rays, dental x-rays, abdominal x-rays, skeletal plain films, computed tomography (CT) scanning, urologic imaging, and mammography; radionuclide imaging, diagnostic fluoroscopy (upper GI series, cholangiography, barium enema), or prolonged fluoroscopy/interventional radiology (coronary angiography, cerebral angiography and transluminal angioplasty).

For humans who may be episodically exposed, the ideal primary dosing schedule consists of one dose consumed orally one to two hours prior to the anticipated exposure. For medical and dental imaging, such as chest x-rays, dental x-rays, abdominal x-rays, skeletal plain films, CT scanning, urologic imaging, or mammography, the primary dosing schedule consists of one dose administered orally from 30 minutes to one hour before the study. The maintenance dosing schedule consists of from one oral dose 2-4 hours after the study to twice daily oral doses for 14 days. Those who are frequent flyers should take one oral dose of BioShield E1 from 30 minutes to two hours before takeoff and another dose 4-6 hours after reaching destination.

For humans who receive radionuclide imaging or prolonged fluoroscopy/interventional radiology (coronary angiography, cerebral angiography and transluminal angioplasty), the primary dosing schedule consists of one dose administered orally from 30 minutes to one hour before the study. The maintenance dosing schedule consists of from one oral dose 2-4 hours after the study and twice a day (morning and evening) for a 5-day period after the study, up to a total of 14 days after the study, depending on the half-life of the radionuclide employed for the procedure.

For humans who receive diagnostic fluoroscopy (upper GI series, cholangiography, barium enema), the primary dosing schedule consists of one dose administered orally from 30 minutes to one hour before the study. The maintenance dosing schedule consists of from one oral dose 2-4 hours after the study and twice a day (morning and evening) for from a 2-day period after the study, up to a total of 14 days after the study.

The formulations for Bioshield E1 are as follows:

### Formulation for Adults, Ages 14 and Older, Consisting Essentially of:

| | primary dose | |
|---|---|---|
| Vitamin A (retinyl palmitate, fish oil or natural mixed | 5,000 | IU |
| carotenoids) | | |
| Vitamin C (calcium ascorbate, citrus, rose or berry | 500 | mg |
| products) | | |
| Vitamin E (d-alpha tocopheryl succinate and/or d-alpha | 400 | IU |
| tocopherol, vegetable or wheat germ products or natural | | |
| tocopheryl) | | |
| L-cysteine (or natural food sources-e.g. soy extracts, | 200 | mg |
| sesame or flaxseeds, oat bran, wheat germ, Swiss | | |
| cheese, peas or whole grain extracts) | | |
| N-acetylcysteine | 250 | mg |
| R-alpha-lipoic acid (R+, R−, yeast or liver sources) | 30 | mg |
| Selenium (L-selenomethionine, sodium selenite/selenate | 100 | mcg |
| or natural forms) | | |
| Natural mixed carotenoids (sea, algal, plant or other | 15 | mg |
| natural sources) | | |

### Formulation for Children (4-13 Years of Age) Consisting Essentially of:

| | primary dose | |
|---|---|---|
| Vitamin A (retinyl palmitate, fish oil or natural | 2,500 | IU |
| mixed carotenoids) | | |
| Vitamin C (calcium ascorbate, citrus, rose or berry | 150 | mg |
| products) | | |
| Vitamin E (d-alpha tocopheryl succinate and/or | 100 | IU |
| d-alpha tocopherol, vegetable or wheat germ | | |
| products or natural tocopheryl) | | |
| L-cysteine (or natural food sources-e.g. soy extracts, | 100 | mg |
| sesame or flaxseeds, oat bran, wheat germ, Swiss | | |
| cheese, peas or whole grain extracts) | | |
| N-acetylcysteine | 50 | mg |
| R-alpha-lipoic acid (R+, R−, yeast or liver sources) | 15 | mg |
| Selenium (L-selenomethionine, sodium selenite/ | 50 | mcg |
| selenate or natural forms) | | |
| Natural mixed carotenoids (sea, algal, plant or other | 5 | mg |
| natural sources) | | |

The formulation of BioShield E2 is designed for individuals who are workers in or reside near the area of radiation equipment, such as x-ray or gamma-ray machines, nuclear plants, or other technology operated by nuclear power, flight crews (pilots and flight attendants), government installations and military personnel, health care and those who are consistently exposed to radon, smoking, occupational chemicals, pesticides, cellular phone technology, air pollution and biological agents. It is also relevant for proactive consumers who utilize emergency survival kits.

For individuals who are occupationally or consistently exposed to these environments, the primary dosing schedule consists of one dose consumed orally twice a day (morning and evening) for the entire lifespan. In some circumstances of particularly high chronic exposure levels, it may be appropriate for certain individuals to additionally consume the “supplemental booster” list of compounds listed with the BioShield E3 formulation.

The formulation2 for Bioshield E2 is as follows:

### Environmental-Daily, E2 Formulation for Adults, Ages 14 and Older

| | primary dose | |
|---|---|---|
| Vitamin A (retinyl palmitate, fish oil or natural mixed | 3,000 | IU |
| carotenoids) | | |
| Vitamin C (calcium ascorbate, citrus, rose or berry | 1,000 | mg |
| products) | | |
| Vitamin E (equally d-alpha tocopheryl succinate and | 400 | IU |
| d-alpha tocopherol acetate, vegetable or wheat germ | | |
| products or natural tocopheryl) | | |
| L-cysteine (or natural food sources-e.g. soy extracts, | 200 | mg |
| sesame or flaxseeds, oat bran, wheat germ, Swiss | | |
| cheese, peas or whole grain extracts) | | |
| N-acetylcysteine | 250 | mg |
| Vitamin D (cholecalciferol-D3 or fish oils) | 800 | IU |
| Vitamin B1 (thiamine mononitrate or yeast sources) | 4 | mg |
| Vitamin B2 (riboflavin or yeast sources) | 5 | mg |
| Vitamin B3 (niacinamide, nicotinic acid, nicotinamide | 30 | mg |
| or yeast sources) | | |
| Vitamin B6 (pyridoxine or yeast sources) | 5 | mg |
| Folic acid (folate, yeast or liver source) | 800 | mcg |
| Vitamin B12 (cyanocobalamin, methylcobalamin or | 10 | mcg |
| yeast sources) | | |
| Biotin (d-biotin or liver sources) | 200 | mcg |
| Pantothenic acid (d-calcium pantothenate, rice bran, | 10 | mg |
| yeast or liver sources) | | |
| Calcium (citrate, ascorbate, lactate or plant based | 250 | mg |
| sources) | | |
| Magnesium (citrate, lactate, gluconate, oxide, | 125 | mg |
| carbonate, hydroxide, chloride, sulphate, lactic acid, | | |
| Jay phosphate, tribasic phosphate or natural forms) | | |
| Zinc (glycinate, gluconate, oxide, sulphate or natural | 15 | mg |
| forms) | | |
| Selenium (L-selenomethionine, sodium selenite/ | 200 | mcg |
| selenate or natural forms) | | |
| Chromium (picolinate, chromic chloride or natural | 50 | mcg |
| forms) | | |
| Coenzyme Q10 (ubiquinone, organ meat or natural | 30 | mg |
| ingredient sources-e.g. Agrobacterium umefaciens, | | |
| Paracoccus denitrificans or Pseudomonas | | |
| aeruginosa) | | |
| R-alpha-lipoic acid (R+, R−, yeast or liver sources) | 30 | mg |
| L-carnitine (fumarate or natural forms) | 100 | mg |
| Natural mixed carotenoids (sea, algal, plant or other | 15 | mg |
| natural sources) | | |

No iron, copper or manganese would be included because these trace minerals are known to interact with vitamin C to produce free radicals. These minerals are also absorbed more readily from the intestinal tract in the presence of antioxidants, and this could result in potentially harmful increased body stores of unbound minerals.

BioShield E3 is recommended for individuals exposed to high, or potentially lethal doses of hazardous environments (e.g. pollution levels, biological agents, space travel, government, military) or those related to unexpected events, such as nuclear accidents (e.g. Chernobyl nuclear power plant leak, Russia, 1986; Fukushima Daiichi nuclear power plant weather-related damage, Japan, 2011) or deliberate terrorist attacks. In ionizing radiation exposure, these doses are associated with development of acute radiation sickness (ARS), represented by bone marrow syndrome (adverse effects on blood cells) that can cause up to 100% lethality within 60 days in humans, depending upon the dose; gastrointestinal syndrome (adverse symptoms of intestinal injury) that can cause 100% lethality within 14 days in humans; and central nervous system syndrome (adverse neurological effects) that can cause 100% lethality within 24 hours. The survivors of these high radiation doses have increased risk of cancerous and non-cancerous diseases and heritable mutations that appear in future generations.

BioShield E3 is intended to be consumed daily for a limited time period depending on the level and severity of exposure. For those who develop diagnosed serious medical conditions as a result of the exposure, it is to be used as an adjunct to standard therapy that may include fluid and electrolyte replacement, antibiotics, blood transfusion, growth factors, such as granulocyte colony-stimulating factor, and consideration of bone marrow or stem cell transplantation.

The BioShield E3 formulation should be taken orally and divided into two doses, half in the morning and the other half in the evening preferably with meals. This is because the biological half-lives of micronutrients (water-soluble and fat-soluble) are highly variable which can create marked fluctuations in tissue levels, differences in gene profile expression and cellular stress.

Consumption of the formulation should be started 24 hours after exposure and continued for up to 60 days depending on the judgment of the health care provider. The 24-hour post-exposure waiting period is desirable because immediately after exposure, pro- and anti-inflammatory cytokines are released in response to cellular injuries. During this period, the proportion of anti-inflammatory cytokines responsible for repair of injury may be higher than that of pro-inflammatory cytokines that further damage cells and tissues. Inhibition of the inflammatory response by the high doses of antioxidants too soon after exposure may prevent the release of both anti- and pro-inflammatory cytokines, and thereby prevent the repair of injury. However, after 24 hours, pro-inflammatory cytokines are expected to dominate. Therefore, inhibition of inflammation at this time may help to prevent progression of damage. The 60-day consumption period is appropriate because by this time, those who would succumb to the exposure will have likely died. Since survivors of will have increased risk of neoplastic and non-neoplastic diseases, it is recommended that when the BioShield R3 supplementation has stopped after 60 days, the BioShield R2 formulation should be consumed twice daily for the remainder of the individual's lifespan in order to reduce the risk of the late adverse health effects of hazardous exposures.

Hazardous environmental exposures are implicated in many adverse health conditions worldwide and antioxidants are protective agents involving many cellular processes that must be considered as targets for therapeutic intervention (1). The present invention relates to the use of multiple micronutrients including dietary and endogenous antioxidants with glutathione-elevating agents to reduce the adverse health effects on humans during episodic, occupational or accidental exposures to hazardous environmental factors or potentially toxic agents. This includes the relevant affected groups listed in Sections 1.1, 2.1 and 3.1 above. In addition to the oral mode of consumption that is outlined in Sections 1.2, 2.2 and 3.3 above, the BioShield formulations may be provided in several alternative platforms including, but not limited to liquids, aerosols, dissolvable disks, injectable forms, those absorbed through the skin, lipid-encased and droplet forms and delayed absorption technologies.

The formulation for Bioshield E3 is as follows:

### BioShield Environmental-Catastrophic, E3 for Adults, Ages 14 Years and Older

| | primary dose | |
|---|---|---|
| Vitamin A (retinyl palmitate, fish oils or natural | 3,000 | IU |
| mixed carotenoids) | | |
| Vitamin C (calcium ascorbate, citrus, rose or berry | 1,000 | mg |
| products) | | |
| Vitamin E (2:1 ratio: d-alpha tocopheryl succinate to | 400 | IU |
| d-alpha tocopherol acetate, vegetable or wheat germ | | |
| products or natural tocopheryl) | | |
| L-cysteine (or natural food sources-e.g. soy extracts, | 400 | mg |
| sesame or flaxseeds, oat bran, wheat germ, Swiss | | |
| cheese, peas or whole grain extracts) | | |
| N-acetylcysteine | 500 | mg |
| Vitamin D (as cholecalciferol-D3 or fish oils) | 800 | IU |
| Vitamin B1 (thiamine mononitrate or yeast sources) | 4 | mg |
| Vitamin B2 (riboflavin or yeast sources) | 5 | mg |
| Vitamin B3 (niacinamide, nicotinic acid, | 30 | mg |
| nicotinamide or yeast sources) | | |
| Vitamin B6 (pyridoxine or yeast sources) | 5 | mg |
| Folic acid (folate, yeast or liver sources) | 800 | mcg |
| Vitamin B12 (cyanocobalamin, methylcobalamin or | 10 | mcg |
| yeast sources) | | |
| Biotin (d-biotin or liver sources) | 200 | mcg |
| Pantothenic acid (d-calcium pantothenate, rice bran, | 10 | mg |
| yeast or liver sources) | | |
| Calcium (citrate, ascorbate, lactate or plant based | 250 | mg |
| sources) | | |
| Magnesium (citrate, lactate, gluconate, oxide, | 125 | mg |
| carbonate, hydroxide, chloride, sulphate, lactic acid, | | |
| Jay phosphate, tribasic phosphate or natural forms) | | |
| Zinc (glycinate, gluconate, oxide, sulphate or natural | 15 | mg |
| forms) | | |
| Selenium (L-selenomethionine, sodium selenite/ | 200 | mcg |
| selenate or natural forms) | | |
| Chromium (picolinate, chromic chloride or natural | 50 | mcg |
| forms) | | |
| Coenzyme Q10 (ubiquinone, organ meat or natural | 45 | mg |
| ingredient sources-e.g. Agrobacterium tumefaciens, | | |
| Paracoccus denitrificans, Pseudomonas | | |
| aeruginosa) | | |
| R-alpha-lipoic acid (R+, R−, yeast or liver sources) | 90 | mg |
| Natural mixed carotenoids (sea, algal or plant | 60 | mg |
| sources) | | |
| L-carnitine (fumarate or natural forms) | 200 | mg |

Numerous modifications and variations of the present invention are possible in light of the above teachings. It is therefore to be understood that within the scope of the attendant claims attached hereto, this invention may be practiced otherwise than as specifically disclosed herein.

### REFERENCES

1. Okunieff, P., Swarts, S., Keng, P., Sun, W., Wang, W., Kim, J., Yang, S., Zhang, H., Liu, C., Williams, J. P., Huser, A. K., Zhang, L. Antioxidants reduce consequences of radiation exposure. Adv Exo Med Biol, 614: 165-178, 2008.
2. Roston, B. L., Chang, C. M., Pechacek, T. F. Estimation of Cigarette Smoking-Attributable Morbidity in the United States. JAMA Intern Med doi: 10.1001/jama intern med. 2014. 5219. Epub Oct. 13, 2014.
3. Coppes, Z., De Vecchi, S., Coppes, P., Prasad, K. A multiple micronutrient supplement increases immune competency and decreases oxidative stress in healthy volunteers: a pilot study. Montevideo, Uruguay: University of Uruguay; 2006.
4. Hodgdon, J., Prasad, K. N., Cole, W. C. Beneficial effects of micronutrients on oxidative damage during intense military training. San Diego (Calif.): Naval Health Research Center; 2008.
5. Gottshall, K. R., Hoffer, M. E., Balough, B. J. Characterizing vestibular deficits and function after blast injury; effects of antioxidant intervention. Presented at the 24^{th} meeting, the Barany Society. Sweden: Uppsala; 2006.
6. Kleinerman, R. A. Cancer risks following diagnostic and therapeutic radiation exposure in children. Pediatr Radiol, 36 Suppl 14: 121-125, 2006.
7. Prasad, K. N. Handbook of Radiobiology, Second Edition. CRC Press, Boca Raton, Fla., 1995.
8. Shah, N. B. and Platt, S. L. ALARA: is there a cause for alarm? Reducing radiation risks from computed tomography scanning in children. Curr Opin Pediatr, 20: 243-247, 2008.
9. ICRP 1990 recommendations of the International Commission on radiological protection. Vol. 21, pp. 1-3: Ananals of the ICRP, 1991.
10. Hall, E. J. Radiation biology for pediatric radiologists. Pediatr Radiol, 2008.
11. Brody, A. S., Frush, D. P., Huda, W., and Brent, R. L. Radiation risk to children from computed tomography. Pediatrics, 120: 677-682, 2007.
12. Chodick, G., Ronckers, C. M., Shalev, V., and Ron, E. Excess lifetime cancer mortality risk attributable to radiation exposure from computed tomography examinations in children. Isr Med Assoc J, 9: 584-587, 2007.
13. Brenner, D. J. and Sachs, R. K. Estimating radiation-induced cancer risks at very low doses: rationale for using a linear no-threshold approach. Radiat Environ Biophys, 44: 253-256, 2006.
14. Radiation, N. R. C. o. t. N. A. C. t. A. H. R. f E. t. L. L. o. i. BEIR VII,

Phase 2. The National Academies Press, Washington, D.C., 2006.
15. Robbins, E. Radiation risks from imaging studies in children with cancer. Pediatr Blood Cancer, 51: 453-457, 2008.
16. Hall, E., J. Garcia, A J Radiobiology for Radiologist. Lippincott Williams & wilkins, Philadelphia, 2006.
17. Rice, H. E., Frush, D. P., Farmer, D., and Waldhausen, J. H. Review of radiation risks from computed tomography: essentials for the pediatric surgeon. J Pediatr Surg, 42: 603-607, 2007.
18. Hall, E. J. Lessons we have learned from our children: cancer risks from diagnostic radiology. Pediatr Radiol, 32: 700-706, 2002.
19. Brenner, D., Elliston, C., Hall, E., and Berdon, W. Estimated risks of radiation-induced fatal cancer from pediatric CT. AJR Am J Roentgenol, 176: 289-296, 2001.
20. Wan, M. J., Krahn, M., Ungar, W. J., Caku, E., Sung, L., Medina, L. S., and Doria, A. S. Acute Appendicitis in Young Children: Cost-effectiveness of US versus CT in Diagnosis—A Markov Decision Analytic Model. Radiology, 2008.
21. Picano, E., Vano, E., Semelka, R., and Regulla, D. The American College of Radiology white paper on radiation dose in medicine:deep impact on the practice of cardiovascular imaging. Cardiovasc Ultrasound, 5: 37, 2007.
22. Picano, E. Risk-benefit balance in cardiovascular imaging: the radiation issue. G Ital Cardiol (Rome), 9: 808-814, 2008.
23. Berrington de Gonzalez, A. and Darby, S. Risk of cancer from diagnostic X-rays: estimates for the UK and 14 other countries. Lancet, 363: 345-351, 2004.
24. Bedetti, G., Botto, N., Andreassi, M. G., Traino, C., Vano, E., and Picano,

E. Cumulative patient effective dose in cardiology. Br J Radiol, 81: 699-705, 2008.
25. Amis, E. S., Jr., Butler, P. F., Applegate, K. E., Birnbaum, S. B., Brateman, L. F., Hevezi, J. M., Mettler, F. A., Morin, R. L., Pentecost, M. J., Smith, G. G., Strauss, K. J., and Zeman, R. K. American College of Radiology white paper on radiation dose in medicine. J Am Coll Radiol, 4: 272-284, 2007.
26. Andreassi, M. G., Cioppa, A., Manfredi, S., Palmieri, C., Botto, N., and Picano, E. Acute chromosomal DNA damage in human lymphocytes after radiation exposure in invasive cardiovascular procedures. Eur Heart J, 28: 2195-2199, 2007.
27. BEIR VII, p. Health risks from exposure to low levels of ionizing radiation. Washington D.C.: The national Academies Press, 2006.
28. Mettler, F. A., Jr., Huda, W., Yoshizumi, T. T., and Mahesh, M. Effective doses in radiology and diagnostic nuclear medicine: a catalog. Radiology, 248: 254-263, 2008.
29. Angelieri, F., de Oliveira, G. R., Sannomiya, E. K., and Ribeiro, D. A., DNA damage and cellular death in oral mucosa cells of children who have undergone panoramic dental radiography, Pediatr Radiol 37 (6), 561-5, 2007.
30. Ribeiro, D. A. and Angelieri, F., Cytogenetic biomonitoring of oral mucosa cells from adults exposed to dental X-rays, Radiat Med 26 (6), 325-30, 2008.
31. Holland, N., Bolognesi, C., Kirsch-Volders, M., Bonassi, S., Zeiger, E., Knasmueller, S., and Fenech, M. The micronucleus assay in human buccal cells as a tool for biomonitoring DNA damage: the HUMN project perspective on current status and knowledge gaps. Mutat Res, 659: 93-108, 2008.
32. Hamzany, Y., Feinmesser, R., Shpitzer, T., Mizrachi, A., Hilly, O., Hod, R., Bahar, G., Otradnov, I., Gavish, M., Nagler, R. M. Is human saliva an indicator of the adverse health effects of using mobile phones? Antioxidants Redox Signal 18 (6): 622, 2013. doi: 10.1089/ars.2012.4751
33. Desai, N. R., Kesari, K. K., Agarwal, A. Pathophysiology of cell phone radiation: oxidative stress and cardinogenesis with focus on male reproductive system. Reproductive Biol Endocrinol 7:114-122, 2009
34. Han, Y. Y., Kano, H., Davis, D. L., Niranjan, A., and Lunsford, L. D., Cell phone use and acoustic neuroma: the need for standardized questionnaires and access to industry data, Surg Neurol 72 (3): 216-222, 2009.
35. Hardell, L. and Sage, C., Biological effects from electromagnetic field exposure and public exposure standards, Biomed Pharmacother 62 (2), 104-9, 2008.
36. Hardell, L., Carlberg, M., Soderqvist, F., Mild, K. H., and Morgan, L. L., Long-term use of cellular phones and brain tumours: increased risk associated with use for > or =10 years, Occup Environ Med 64 (9), 626-32, 2007.
37. Hardell, L., Carlberg, M., Soderqvist, F., and Hansson Mild, K., Meta-analysis of long-term mobile phone use and the association with brain tumours, Int J Oncol 32 (5), 1097-103, 2008.
38. Takebayashi, T., Akiba, S., Kikuchi, Y., Taki, M., Wake, K., Watanabe, S., and Yamaguchi, N., Mobile phone use and acoustic neuroma risk in Japan, Occup Environ Med 63 (12), 802-7, 2006.
39. Croft, R. J., McKenzie, R. J., Inyang, I., Benke, G. P., Anderson, V., and

Abramson, M. J., Mobile phones and brain tumours: a review of epidemiological research, Australas Phys Eng Sci Med 31 (4), 255-67, 2008.
40. Sadetzki, S., Chetrit, A., Jarus-Hakak, A., Cardis, E., Deutch, Y., Duvdevani, S., Zultan, A., Novikov, I., Freedman, L., and Wolf, M., Cellular phone use and risk of benign and malignant parotid gland tumors—a nationwide case-control study, Am J Epidemiol 167 (4), 457-67, 2008.
41. Abdel-Rassoul, G., El-Fateh, O. A., Salem, M. A., Michael, A., Farahat, F., El-Batanouny, M., and Salem, E., Neurobehavioral effects among inhabitants around mobile phone base stations, Neurotoxicology 28 (2), 434-40, 2007.
42. Tillmann, T., Ernst, H., Ebert, S., Kuster, N., Behnke, W., Rittinghausen, S., and Dasenbrock, C., Carcinogenicity study of GSM and DCS wireless communication signals in B6C3F1 mice, Bioelectromagnetics 28 (3), 173-87, 2007.
43. Kim, J. Y., Hong, S. Y., Lee, Y. M., Yu, S. A., Koh, W. S., Hong, J. R., Son, T.,

Chang, S. K., and Lee, M., In vitro assessment of clastogenicity of mobile-phone radiation (835 MHz) using the alkaline comet assay and chromosomal aberration test, Environ Toxicol 23 (3), 319-27, 2008.
44. Buja, A., Lange, J. H., Perissinotto, E., Rausa, G., Grigoletto, F., Canova, C., and Mastrangelo, G. Cancer incidence among male military and civil pilots and flight attendants: an analysis on published data. Toxicol Ind Health, 21: 273-282, 2005.
45. Band, P. R., Le, N. D., Fang, R., Deschamps, M., Coldman, A. J., Gallagher, R. P., and Moody, J. Cohort study of Air Canada pilots: mortality, cancer incidence, and leukemia risk. Am J Epidemiol, 143: 137-143, 1996.
46. Gunderstrup, M., Storm, H. H. Radiation-induced acute myeloid leukemia and other cancers in commercial jet cockpit crew: a population-based cohort study. Lancet 354: 2029-2031, 1999.
47. Ballard, T., Lagorio, S., De Angelis, G., Verdecchia, A. Cancer incidence and mortality among flight personnel: a meta-analysis. Aviation Space Environment Med 71: 216-224, 2000.
48. Sigurdson, A. J. and Ron, E. Cosmic radiation exposure and cancer risk among flight crew. Cancer Invest, 22: 743-761, 2004.
49. Rafnsson, V., Tulinius, H., Jonasson, J. G., and Hrafnkelsson, J. Risk of breast cancer in female flight attendants: a population-based study (Iceland). Cancer Causes Control, 12: 95-101, 2001.
50. Pukkala, E., Auvinen, A., and Wahlberg, G. Incidence of cancer among Finnish airline cabin attendants, 1967-92. BMJ, 311: 649-652, 1995.
51. Grayson, J. K., Lyons, T. J. Cancer incidence in United States Air Force aircrew, 1975-89. Aviation Space Environment Med 67: 101-104, 1996
52. Heimers, A. Chromosome aberration analysis in Concorde pilots. Mutation Res 467:169-176, 2000.
53. Romano, E., Ferrucci, L., Nicolai, F., Derme, V., De Stefano, G. F. Increase of chromosomal aberrations induced by ionizing radiation in peripheral blood lymphocytes of civil aviation pilots and crew members. Mutation Res 377: 89-93, 1997.
54. Ben-Amotz, A., Yatziv, S., Sela, M., Greenberg, S., Rachmilevich, B., Shwarzman, M., and Weshler, Z. Effect of natural beta-carotene supplementation in children exposed to radiation from the Chernobyl accident. Radiat Environ Biophys, 37: 187-193, 1998.
55. Korkina, L. G., Afanas′ef, I. B., and Diplock, A. T. Antioxidant therapy in children affected by irradiation from the Chernobyl nuclear accident. Biochem Soc Trans, 21 (Pt 3): 314S, 1993.
56. Unreleased Federal Study, U.S. Government, reported in USA Today, 2005.
57. Stone, R. Russian cancer study adds to the indictment of low-dose radiation. Science 310:959, 2005.
58. Mangano, J., Sherman, J., Busby, C. Changes in confirmed plus borderline cases of congenital hypothyroidism in California as a function of environmental fallout from the Fukushima nuclear meltdown. O J Pediatr 3 (4): 370-376, 2013. doi:10.4236/ojped.2013.34067
59. Frumkin, H, Samet, J. M., Radon. CA Cancer J Clin 51: 337-344, 2001.
60. Peacock, M, Radon gas more deadly than carbon monoxide poisoning. U.S. Environmental Protection Agency, news release, January, 2008.
61. Wheeler, B. W., Skin cancer rate may be higher in high-radon areas. Epidemiology Online, Nov. 10, 2011.
62. Kelly, F. J., Oxidative stress: its role in air pollution and adverse health effects. Occup Environ Med 60: 612-616, 2003.
63. Kuehn, B. M., WHO: more than 7 million air pollution deaths each year. JAMA 311: 1486, 2014.
64. Patz, J. A, Frumkin, H., Holloway, T., Vimont, D. J., Haines, A. Climate change: challenges and opportunities for global health. JAMA 312: 1565-1580, 2014.
65. Perera, F., Faraone, S. ADHD linked to expectant mom's smog exposure. Pub Libr Sci PLoS One, Online, Nov. 5, 2014.
66. Hashemi, N., Boskabady, M. H., and Nazari, A. Occupational Exposures and Obstructive Lung Disease: A Case-Control Study in Hairdressers. Respir Care, 55 (7): 895-900, 2010.
67. Borek, C., Ong, A., Mason, H., Donahue, L., and Biaglow, J. E. Selenium and vitamin E inhibit radiogenic and chemically induced transformation in vitro via different mechanisms. Proc Natl Acad Sci USA, 83: 1490-1494, 1986.
68. Radner, B. S. and Kennedy, A. R. Suppression of X-ray induced transformation by vitamin E in mouse C3H/10T1/2 cells. Cancer Lett, 32: 25-32, 1986.
69. Kennedy, A. R. and Krinsky, N. I. Effects of retinoids, beta-carotene, and canthaxanthin on UV- and X-ray-induced transformation of C3H10T1/2 cells in vitro. Nutr Cancer, 22: 219-232, 1994.
70. Gaziev, A. I., Podlutsky, A., Panfilov, B. M., and Bradbury, R. Dietary supplements of antioxidants reduce hprt mutant frequency in splenocytes of aging mice. Mutat Res, 338: 77-86, 1995.
71. Konopacka, M., Widel, M., and Rzeszowska-Wolny, J. Modifying effect of vitamins C, E and beta-carotene against gamma-ray-induced DNA damage in mouse cells. Mutat Res, 417: 85-94, 1998.
72. Kumar, B., Jha, M. N., Cole, W. C., Bedford, J. S., and Prasad, K. N. D-alpha-tocopheryl succinate (vitamin E) enhances radiation-induced chromosomal damage levels in human cancer cells, but reduces it in normal cells. J Am Coll Nutr, 21: 339-343, 2002.
73. Ni, Q. G. and Pei, Y. [Effect of beta-carotene on 60Co-gamma-induced mutation at T-lymphocyte hypoxanthine-guanine phosphoribosyl transferase locus in rats]. Zhongguo Yao Li Xue Bao, 18: 535-536, 1997.
74. O'Connor, M. K., Malone, J. F., Moriarty, M., and Mulgrew, S. A radioprotective effect of vitamin C observed in Chinese hamster ovary cells. Br J Radiol, 50: 587-591, 1977.
75. Okunieff, P., Swarts, S., Keng, P., Sun, W., Wang, W., Kim, J., Yang, S., Zhang, H., Liu, C., Williams, J. P., Huser, A. K., and Zhang, L. Antioxidants reduce consequences of radiation exposure. Adv Exp Med Biol, 614: 165-178, 2008.
76. Ushakova, T., Melkonyan, H., Nikonova, L., Afanasyev, V., Gaziev, A. I., Mudrik, N., Bradbury, R., and Gogvadze, V. Modification of gene expression by dietary antioxidants in radiation-induced apoptosis of mice splenocytes. Free Radic Biol Med, 26: 887-891, 1999.
77. Weiss, J. F. and Landauer, M. R. Radioprotection by antioxidants. Ann N Y Acad Sci, 899: 44-60, 2000.
78. Weiss, J. F. and Landauer, M. R. Protection against ionizing radiation by antioxidant nutrients and phytochemicals. Toxicology, 189: 1-20, 2003.
79. Ramakrishnan, N., Wolfe, W. W., and Catravas, G. N. Radioprotection of hematopoietic tissues in mice by lipoic acid. Radiat Res, 130: 360-365, 1992.
80. Blumenthal, R. D., Lew, W., Reising, A., Soyne, D., Osorio, L., Ying, Z., and Goldenberg, D. M. Anti-oxidant vitamins reduce normal tissue toxicity induced by radio-immunotherapy. Int J Cancer, 86: 276-280, 2000.
81. El-Habit, O. H., Saada, H. N., Azab, K. S., Abdel-Rahman, M., and El-Malah, D. F. The modifying effect of beta-carotene on gamma radiation-induced elevation of oxidative reactions and genotoxicity in male rats. Mutat Res, 466: 179-186, 2000.
82. Ershoff, B. H. and Steers, C. W., Jr. Antioxidants and survival time of mice exposed to multiple sublethal doses of x-irradiation. Proc Soc Exp Biol Med, 104: 274-276, 1960.
83. Harapanhalli, R. S., Yaghmai, V., Giuliani, D., Howell, R. W., and Rao, D. V. Antioxidant effects of vitamin C in mice following X-irradiation. Res Commun Mol Pathol Pharmacol, 94: 271-287, 1996.
84. Mills, E. E. The modifying effect of beta-carotene on radiation and chemotherapy induced oral mucositis. Br J Cancer, 57: 416-417, 1988.
85. Mutlu-Turkoglu, U., Erbil, Y., Oztezcan, S., Olgac, V., Toker, G., and Uysal, M. The effect of selenium and/or vitamin E treatments on radiation-induced intestinal injury in rats. Life Sci, 66: 1905-1913, 2000.
86. Narra, V. R., Harapanhalli, R. S., Howell, R. W., Sastry, K. S., and Rao, D. V. Vitamins as radioprotectors in vivo. I. Protection by vitamin C against internal radionuclides in mouse testes: implications to the mechanism of damage caused by the Auger effect. Radiat Res, 137: 394-399, 1994.
87. Umegaki, K., Uramoto, H., Suzuki, J., and Esashi, T. Feeding mice palm carotene prevents DNA damage in bone marrow and reduction of peripheral leukocyte counts, and enhances survival following X-ray irradiation. Carcinogenesis, 18: 1943-1947, 1997.
88. Seifter E, R., A, Padawar, J, Levenson, S M. Vitamin A abd beta-carotene as adjunctive therapy to tumor excision, radiation therapy and chemotherapy. In: Vitamins, Nutrition and Cancer, Karger, Basel, 1984, pp. 1-19.
89. Guan, J., Wan, X. S., Zhou, Z., Ware, J., Donahue, J. J., Biaglow, J. E., and Kennedy, A. R. Effects of dietary supplements on space radiation-induced oxidative stress in Sprague-Dawley rats. Radiat Res, 162: 572-579, 2004.
90. Kennedy, A. R., Zhou, Z., Donahue, J. J., and Ware, J. H. Protection against adverse biological effects induced by space radiation by the Bowman-Birk inhibitor and antioxidants. Radiat Res, 166: 327-332, 2006.
91. Wan, X. S., Ware, J. H., Zhou, Z., Donahue, J. J., Guan, J., and Kennedy, A. R. Protection against radiation-induced oxidative stress in cultured human epithelial cells by treatment with antioxidant agents. Int J Radiat Oncol Biol Phys, 64: 1475-1481, 2006.
92. Kocer, I., Taysi, S., Ertekin, M. V., Karslioglu, I., Gepdiremen, A., Sezen, O., and Serifoglu, K. The effect of L-carnitine in the prevention of ionizing radiation-induced cataracts: a rat model. Graefes Arch Clin Exp Ophthalmol, 245: 588-594, 2007.
93. Sezen, O., Ertekin, M. V., Demircan, B., Karslioglu, I., Erdogan, F., Kocer, I., Calik, I., and Gepdiremen, A. Vitamin E and L-carnitine, separately or in combination, in the prevention of radiation-induced brain and retinal damages. Neurosurg Rev, 31: 205-213, 2008.
94. Altas, E., Ertekin, M. V., Gundogdu, C., and Demirci, E. L-carnitine reduces cochlear damage induced by gamma irradiation in Guinea pigs. Ann Clin Lab Sci, 36: 312-318, 2006.
95. Sminia, P., van der Kracht, A. H., Frederiks, W. M., and Jansen, W. Hyperthermia, radiation carcinogenesis and the protective potential of vitamin A and N-acetylcysteine. J Cancer Res Clin Oncol, 122: 343-350, 1996.
96. Krinsky, N. I. Antioxidant functions of carotenoids. Free Radic Biol Med, 7: 617-635, 1989.
97. Hazuka, M. B., Edwards-Prasad, J., Newman, F., Kinzie, J. J., and Prasad, K. N. Beta-carotene induces morphological differentiation and decreases adenylate cyclase activity in melanoma cells in culture. J Am Coll Nutr, 9: 143-149, 1990.
98. Zhang, L. X., Cooney, R. V., and Bertram, J. S. Carotenoids up-regulate connexin43 gene expression independent of their provitamin A or antioxidant properties. Cancer Res, 52: 5707-5712, 1992.
99. Carter, C. A., Pogribny, M., Davidson, A., Jackson, C. D., McGarrity, L. J., and Morris, S. M. Effects of retinoic acid on cell differentiation and reversion toward normal in human endometrial adenocarcinoma (RL95-2) cells. Anticancer Res, 16: 17-24, 1996.
100. Meyskens Jr, F. L. Role Of Vitamin A and Its Derivatives In The Treatment Of Human Cancer. In: K. N. Prasad, L. Santamaria, and R. M. Williams (eds.), Nutrients In Cancer Prevention And Treatment, pp. 349-362. New Jersey: Humana Press, 1995.
101. Vile, G. F. and Winterbourn, C. C. Inhibition of adriamycin-promoted microsomal lipid peroxidation by beta-carotene, alpha-tocopherol and retinol at high and low oxygen partial pressures. FEBS Lett, 238: 353-356, 1988.
102. McCay, P. B. Vitamin E: interactions with free radicals and ascorbate. Annu Rev Nutr, 5: 323-340, 1985.
103. Prasad, K. N., Kumar, B., Yan, X. D., Hanson, A. J., and Cole, W. C. alpha-Tocopheryl Succinate, the Most Effective Form of Vitamin E for Adjuvant Cancer Treatment: A Review. J Am Coll Nutr, 22: 108-117, 2003.
104. Schwartz, J. L. Molecular and Biochemical Control of Tumor Growth Following Treatment with Carotenoids or Tocopherols. In: K. N. Prasad, L. Santamaria, and R. M. Williams (eds.), Nutrients in Cancer Prevention and Treatment, pp. 287-316. New Jersey: Humana Press, 1995.
105. Prasad, K. N. and Edwards-Prasad, J. Vitamin E and cancer prevention: recent advances and future potentials. J Am Coll Nutr, 11: 487-500, 1992.
106. Witschi, A., Reddy, S., Stofer, B., and Lauterburg, B. H. The systemic availability of oral glutathione. Eur J Clin Pharmacol, 43: 667-669, 1992.
107. Niki, E. Mechanisms and dynamics of antioxidant action of ubiquinol. Mol Aspects Med, 18 Suppl: S63-70, 1997.
108. Hiramatsu, M., Velasco, R. D., Wilson, D. S., and Packer, L. Ubiquinone protects against loss of tocopherol in rat liver microsomes and mitochondrial membranes. Res Commun Chem Pathol Pharmacol, 72: 231-241, 1991.
109. Abate, A., Yang, G., Dennery, P. A., Oberle, S., and Schroder, H. Synergistic inhibition of cyclooxygenase-2 expression by vitamin E and aspirin. Free Radic Biol Med, 29: 1135-1142, 2000.
110. Devaraj, S. and halal, I. Alpha tocopherol supplementation decreases serum C-reactive protein and monocyte interleukin-6 levels in normal volunteers and type 2 diabetic patients. Free Radic Biol Med, 29: 790-792, 2000.
111. Guenechea, G., Albella, B., Bueren, J. A., Maganto, G., Tuduri, P., Guerrero, A., Pivel, J. P., and Real, A. AM218, a new polyanionic polysaccharide, induces radioprotection in mice when administered shortly before irradiation. Int J Radiat Biol, 71: 101-108, 1997.
112. Patchen, M. L., MacVittie, T. J., Solberg, B. D., D'Alesandro, M. M., and Brook, I. Radioprotection by polysaccharides alone and in combination with aminothiols. Adv Space Res, 12: 233-248, 1992.
113. Ross, W. M. and Peeke, J. Radioprotection conferred by dextran sulfate given before irradiation in mice. Exp Hematol, 14: 147-155, 1986.
114. Kuefner, M. A., Brand, M., Ehrlich, J., Brga, L., Uder, M., and Semelka, C. Effect of antioxidants on X-ray-induced gamma-H2AX foci in human blood lymphocytes: preliminary onservations. Radiology 264: 59-67, 2012.
115. Prasad, K. N. Rationale for using multiple antioxidants in protecting humans against low doses of ionizing radiation. Br J Radiol, 78: 485-492, 2005.

What is claimed is:

1. A formulation consisting essentially of antioxidants, said antioxidants are selected from the group consisting essentially of Vitamin A, Vitamin C, Vitamin E, L-cysteine, N-acetylcysteine, R-alpha-lipoic acid, Selenium, Natural mixed carotenoids and mixtures thereof.

2. The formulation of claim 1 wherein said the formulation comprises at least one glutathione elevating agent.

3. The formulation of claim 1 wherein a dosage level of antioxidants is proportionate to the radiation level exposed to by a human.

4. The formulation of claim 1 wherein said formulation is designed for a human who exposed to an effective dose of ionizing radiation of 0.5 mSv or less, radon, smoking, biological agents, or radiation.

5. The formulation of claim 1 wherein said formulations is for health care, aviation, military, and government employees.

5. The formulation of claim 4 wherein the antioxidants consist essentially of 5,000 IU of Vitamin A, 500 mg of vitamin C, 400 international units of d-alpha tocopheryl acid succinate, 200 mg of L-cyteine, 30 mg of R-alpha-lipoic acid, and 250 mg of N-acetyl cysteine, 100 mcg of Selenium, and 15 mg of natural mixed carotenoids.

6. The formulation of claim 1 wherein said formulation is designed for a human who is consistently exposed to an effective dose of ionizing radiation of 0.5-5 mSv, radon, smoking, occupational chemicals, pesticides, cellular phone technology, air pollution and biological agents.

7. The formulation of claim 6 wherein the antioxidants consist essentially of 3,00 IU of Vitamin A, 1,000 mg of buffered vitamin C, 400 international units of d-alpha tocopheryl acid succinate, 200 mg of L-cysteine, 250 mg of N-acetyl cysteine, 800 IU of Vitamin D, 4 mg of Vitamin B1, 5 mg of Vitamin B2, 30 mg of Vitamin B3, 5 mg of Vitamin B6, 800 mcg of Folic acid, 10 mcg of Vitamin B12, 200 mcg of Biotin, 10 mg or Pantothenic acid, 250 mg calcium, 125 mg of Magnesium, 15 mg of Zinc, 200 mcg of Selenium, 50 mcg of Chromium, 30 mg R-alpha-lipoic acid, 100 mg of L-carnitine, and 15 mg of natural mixed carotenoids.

8. The formulation of claim 1 wherein said formulation is designed for a human who receives an effective dose of ionizing radiation of 5-250 mSv, nuclear accidents, terror attacks, or potentially lethal doses of radiation.

9. The formulation of claim 8 wherein said antioxidants consist essentially of 3,00 IU of Vitamin A, 1,000 mg of buffered vitamin C, 400 international units of d-alpha tocopheryl acid succinate, 200 mg of L-cysteine, 250 mg of N-acetyl cysteine, 800 IU of Vitamin D, 4 mg of Vitamin B1, 5 mg of Vitamin B2, 30 mg of Vitamin B3, 5 mg of Vitamin B6, 800 mcg of Folic acid, 10 mcg of Vitamin B12, 200 mcg of Biotin, 10 mg or Pantothenic acid, 250 mg calcium, 125 mg of Magnesium, 15 mg of Zinc, 200 mcg of Selenium, 50 mcg of Chromium, 30 mg R-alpha-lipoic acid, 100 mg of L-carnitine, and 15 mg of natural mixed carotenoids.

10. The formulation of claim 1 wherein said antioxidants consist essentially of:

| | | |
|---|---|---|
| Vitamin A (retinyl palmitate, fish oil or natural | 500-5,000 | IU |
| mixed carotenoids) | | |
| Vitamin C (calcium ascorbate, citrus, rose or berry | 50-500 | mg |
| products) | | |
| Vitamin E (d-alpha tocopheryl succinate and/or | 50-400 | IU |
| d-alpha tocopherol, vegetable or wheat germ | | |
| products or natural tocopheryl) | | |
| L-cysteine (or natural food sources-e.g. soy | 0.01-300 | mg |
| extracts, sesame or flaxseeds, oat bran, wheat | | |
| germ, Swiss cheese, peas or whole grain extracts) | | |
| N-acetylcysteine | 0.01-250 | mg |
| R-alpha-lipoic acid (R+, R−, yeast or liver | 5-60 | mg |
| sources) | | |
| Selenium (L-selenomethionine, sodium selenite/ | 25-300 | mcg |
| selenate or natural forms) | | |
| Natural mixed carotenoids (sea, algal, plant or | 0.01-45 | mg |
| other natural sources) | | |

11. The formulation of claim 1 further consisting essentially of Vitamin D, Vitamin B1, Vitamin B2, Vitamin B3, Vitamin B6, Folic Acid, Vitamin B12, Biotin, Pantothenic acid, Calcium, Magnesium, Zinc, Selenium, Chromium, Coenzyme Q10, L-carnetine, and mixtures thereof.

12. The formulation of claim 11 wherein said antioxidants consist essentially of:

| | | |
|---|---|---|
| Vitamin D (cholecalciferol-D3 or fish oils) | 100-4000 | IU |
| Vitamin B1 (thiamine mononitrate or yeast | 1-10 | mg |
| sources) | | |
| Vitamin B2 (riboflavin or yeast sources) | 1-20 | mg |
| Vitamin B3 (niacinamide, nicotinic acid, | 1-100 | mg |
| nicotinamide or yeast sources) | | |
| Vitamin B6 (pyridoxine or yeast sources) | 1-50 | mg |
| Folic acid (folate, yeast or liver source) | 100-2,000 | mcg |
| Vitamin B12 (cyanocobalamin, methylcobalamin | 1-50 | mcg |
| or yeast sources) | | |
| Biotin (d-biotin or liver sources) | 50-600 | mcg |
| Pantothenic acid (d-calcium pantothenate, rice | 1-50 | mg |
| bran, yeast or liver sources) | | |
| Calcium (citrate, ascorbate, lactate or plant based | 50-1,000 | mg |
| sources) | | |
| Magnesium (citrate, lactate, gluconate, oxide, | 25-500 | mg |
| carbonate, hydroxide, chloride, sulphate, lactic | | |
| acid, Jay phosphate, tribasic phosphate or natural | | |
| forms) | | |
| Zinc (glycinate, gluconate, oxide, sulphate or | 2-45 | mg |
| natural forms) | | |
| Selenium (L-selenomethionine, sodium selenite/ | 25-300 | mcg |
| selenate or natural forms) | | |
| Chromium (picolinate, chromic chloride or natural | 10-200 | mcg |
| forms) | | |
| Coenzyme Q10 (ubiquinone, organ meat or natural | 0.01-100 | mg |
| ingredient sources-e.g. Agrobacterium | | |
| tumefaciens, Paracoccus denitrificans or | | |
| Pseudomonas aeruginosa) | | |
| L-carnitine (fumarate or natural forms) | 20-400 | mg |

13. A method of manufacturing a formulation, said method comprising admixing antioxidants, said antioxidants consist essentially of:

| | | |
|---|---|---|
| Vitamin A (retinyl palmitate, fish oil or natural | 500-5,000 | IU |
| mixed carotenoids) | | |
| Vitamin C (calcium ascorbate, citrus, rose or berry | 50-500 | mg |
| products) | | |
| Vitamin E (d-alpha tocopheryl succinate and/or | 50-400 | IU |
| d-alpha tocopherol, vegetable or wheat germ | | |
| products or natural tocopheryl) | | |
| L-cysteine (or natural food sources-e.g. soy | 0.01-300 | mg |
| extracts, sesame or flaxseeds, oat bran, wheat | | |
| germ, Swiss cheese, peas or whole grain extracts) | | |
| N-acetylcysteine | 0.01-250 | mg |
| R-alpha-lipoic acid (R+, R−, yeast or liver sources) | 5-60 | mg |
| Selenium (L-selenomethionine, sodium selenite/ | 25-300 | mcg |
| selenate or natural forms) | | |
| Natural mixed carotenoids (sea, algal, plant or | 0.01-45 | mg |
| other natural sources) | | |

14. The method of claim 13 wherein a dosage level of antioxidants is proportionate to the radiation level exposed to by a human.

15. A method of manufacturing the formulation of claim 14, said method comprising admixing antioxidants, said antioxidants consist essentially of:

| | | |
|---|---|---|
| Vitamin D (cholecalciferol-D3 or fish oils) | 100-4000 | IU |
| Vitamin B1 (thiamine mononitrate or yeast | 1-10 | mg |
| sources) | | |
| Vitamin B2 (riboflavin or yeast sources) | 1-20 | mg |
| Vitamin B3 (niacinamide, nicotinic acid, | 1-100 | mg |
| nicotinamide or yeast sources) | | |
| Vitamin B6 (pyridoxine or yeast sources) | 1-50 | mg |
| Folic acid (folate, yeast or liver source) | 100-2,000 | mcg |
| Vitamin B12 (cyanocobalamin, methylcobalamin | 1-50 | mcg |
| or yeast sources) | | |
| Biotin (d-biotin or liver sources) | 50-600 | mcg |
| Pantothenic acid (d-calcium pantothenate, rice | 1-50 | mg |
| bran, yeast or liver sources) | | |
| Calcium (citrate, ascorbate, lactate or plant based | 50-1,000 | mg |
| sources) | | |
| Magnesium (citrate, lactate, gluconate, oxide, | 25-500 | mg |
| carbonate, hydroxide, chloride, sulphate, | | |
| lactic acid, Jay phosphate, tribasic phosphate or | | |
| natural forms) | | |
| Zinc (glycinate, gluconate, oxide, sulphate or | 2-45 | mg |
| natural forms) | | |
| Selenium (L-selenomethionine, sodium selenite/ | 25-300 | mcg |
| selenate or natural forms) | | |
| Chromium (picolinate, chromic chloride or | 10-200 | mcg |
| natural forms) | | |
| Coenzyme Q10 (ubiquinone, organ meat or | 0.01-100 | mg |
| natural ingredient sources-e.g. Agrobacterium | | |
| tumefaciens, Paracoccus denitrificans or | | |
| Pseudomonas aeruginosa) | | |
| L-carnitine (fumarate or natural forms) | 20-400 | mg |

16. The method of claim 15 wherein a dosage level of antioxidants is proportionate to the radiation level exposed to by a human.
